(19) United States
(12) Patent Application Publication
(10) Pub. No.: US 2025/0057545 A1
(43) Pub. Date: Feb. 20, 2025

(54) IMPLANTABLE TRAPEZIUM PROSTHETIC AND METHODOLOGY FOR TREATMENT OF ARTHRITIS IN THE THUMB CMC JOINT

(71) Applicant: UNM Rainforest Innovations, Albuquerque, NM (US)

(72) Inventors: Nathan MORRELL, Albuquerque, NM (US); Dimitri MADDEN, Albuquerque, NM (US); Lauren A. OSTERMANN, Albuquerque, NM (US); Diego Enrique RODRIGUEZ, Albuquerque, NM (US); August William FINKE, Albuquerque, NM (US); Ethan C. DARWIN, Albuquerque, NM (US)

(21) Appl. No.: 18/718,713

(22) PCT Filed: Dec. 13, 2022
(86) PCT No.: PCT/US2022/052754

§ 371 Date: Jun. 11, 2024

Related U.S. Application Data
(60) Provisional application No. 63/289,115, filed on Dec. 13, 2021.

(51) Int. Cl. A61B 17/16; A61F 2/30; A61F 2/42

(52) U.S. Cl. CPC A61B 17/1686; A61F 2002/30387; A61F 2002/4253; A61F 2002/4258

(57) ABSTRACT

A procedure and device that resolves CMC arthritis by mimicking a thumb CMC joint fusion, while avoiding common shortcomings of the fusion surgery as well as existing alternative surgeries for thumb CMC arthritis which can also replace the trapezium bone in the carpometacarpal joint with a stemmed trapezial implant. In addition, the trapezial implant device replicates skeletal anatomy and is configured to directly contact native bone and cartilage in order to more evenly distribute stresses.

### RELATED APPLICATIONS

This application claims priority to U.S. Provisional Application No. 63/289,115, filed on 13 Dec. 2022, which is incorporated herein in its entirety.

### BACKGROUND OF THE INVENTION

Thumb carpometacarpal (CMC) arthritis is a common affliction at the joint between the first metacarpal and the trapezium carpal bone. This is the joint where the first metacarpal of the thumb mates with the respective carpal bone of the wrist. The CMC joint is complex because it has multiple rotational axes. The first metacarpal has a saddle-shaped geometry where it rests on the trapezium. Motion at this joint is dependent on the saddle shape between the first metacarpal and the trapezium. The first metacarpal rotates and translates on the trapezium about axes of rotation that are transverse to the saddles. The trapezium is the carpal bone between the first metacarpal bone, the scaphoid carpal bone, and the trapezoid carpal bone.

Thumb CMC arthritis occurs with aging or sometimes with damage when the cartilage at the trapezium and the first metacarpal wears away. The result of arthritis is roughening of the surface of the joint, sometimes to the point of bone-on-bone contact, all causing increased friction. Patients can experience swelling, pain, stiffness, decreased range of motion, and decreased grip strength. In less advanced arthritic stages, splints or anti-inflammatory medication may be used to reduce pain in the joint. However, in thumb CMC arthritis the trapezium bone can become especially damaged and inflamed, deteriorating to advanced stages of arthritis quickly and requiring the assistance of orthopedic specialists and surgical repair.

Existing surgical procedures and implants used for resolving pain and mobility issues from thumb CMC joint arthritis have many shortcomings. These include reduced grip strength, reduced range of motion, painful and lengthy recoveries, challenging procedures for device installation, and high failure rates. The most common surgical procedure for thumb CMC joint arthritis is Ligament Reconstruction and Tendon Interposition (LRTI) surgery. In this procedure, the entire trapezium is removed from the patient's hand. The Flexor Carpi Radialis (FCR) tendon, used in the motion of flexing the wrist, is cut. The length of the tendon is used to stabilize the first metacarpal and fill the gap left by the missing trapezium bone. This surgery is successful in that it reduces pain for patients and restores mobility that was lost from arthritis. However, grip strength is reduced significantly due to the shortening of the thumb and removal of the trapezium. Many devices have been invented to improve the surgical reconstruction of arthritic CMC joints, but these devices have not been highly successful. Some devices have attempted to maintain CMC joint articulation using a stem inserted in the first metacarpal and extending to a ball and socket joint at the trapezium. The ball and socket can be achieved by reshaping the bone or adhering a socket directly to the trapezium. These devices typically fail due to several issues including loss of implant adherence to the trapezium and instability or uncoupling of the implant components. However, the most common reason surgical implants for this joint fail is instability, due to the highly unconstrained nature of the thumb CMC saddle joint. The thumb CMC joint is a highly unconstrained joint with a complex range of motion due to the saddle joint. Unconstrained devices that try to maintain articulation at the CMC joint typically fail. Consultations with hand and wrist orthopedic specialists revealed that many of these shortcomings make current surgical devices unpreferable.

There is an existing surgical procedure that presents solutions to several of the CMC joint repair challenges: Thumb CMC Arthrodesis, a fusion surgery. This surgery resolves CMC arthritis by fusing the trapezium to the first metacarpal. This surgery maintains thumb length and preserves tendon form and tension in the CMC joint area, allowing patients to maintain grip strength. As the surgery constrains the CMC joint, motion is shifted to being through the more proximal joint, the scaphotrapeziotrapezoid joint. Unfortunately, CMC arthrodesis is extremely limiting to the thumb range of motion, where most patients can expect significant reductions in the thumb range of motion. The surgical procedure itself is not trivial, and patient recovery times are extensive, typically requiring several months before the bones have fully fused.

There have been numerous attempts to develop two-piece implants that were meant to recreate an articulating joint in order to address the issues with hard implants and silicone implant deterioration. Early designs frequently consisted of a ball and socket joint on a single stem, which makes surgery more difficult and intrusive by necessitating the removal or contouring of many bones. The CMC joint reconstruction devices have not all been met with a sufficient level of success. Long-term deterioration, loosening, or dislocation tend to be problems.

It is, therefore, necessary to develop a stemmed trapezium bone implant, system, and technique of use for the implantation that addresses some or all of the previously listed shortcomings of earlier carpometacarpal joint implants.

### BRIEF SUMMARY OF THE INVENTION

In order to treat the diseased joint and get around the drawbacks of the previously known devices, this invention aims to offer the surgeon surgical options and the appropriate equipment.

In one aspect, the present invention concerns a procedure and device that resolves CMC arthritis by mimicking a thumb CMC joint fusion, while avoiding common shortcomings of the fusion surgery as well as existing alternative surgeries for thumb CMC arthritis.

The provision of a novel and practical system for replacing the trapezium bone in the carpometacarpal joint with a modular stemmed trapezial implant is another objective of the invention.

Another objective of the idea is to create a trapezial implant device that replicates skeletal anatomy and is configured to directly contact native bone and cartilage in order to more evenly distribute stresses.

Another objective of the innovation is to offer a trapezial implant tool that enables implant stability at the base of the thumb.

Although the invention is shown and described herein as being embodied in devices, implements, and methods for treating a multiple rotational axes joint, it is not intended to be restricted to merely the features disclosed, as many structural alterations and modifications may be made therein without deviating from the essence of the invention and within the extent and range of equivalents of the claims.

It is to be understood that both the foregoing general description and the following detailed description are exemplary and explanatory only and are not restrictive of the invention, as claimed.

### DESCRIPTION OF FIGURES

A preferred embodiment presented in the images of the accompanying drawings can be referred to in order to have a deeper understanding of the invention. Even though the embodiment shown is merely illustrative of the systems and methods that may be used to implement the invention, the organization and mode of operation of the invention, as well as additional goals and benefits thereof, may be better understood by reference to the drawings and the following description. The drawings are only meant to elucidate and provide examples of the invention, not to limit its scope, which is specifically described in the appended or later amended claims.

Reference is now made to the ensuing drawings in order to gain a fuller grasp of the innovation:

FIG. 1 is a diagram of the first metacarpals, carpal bones, and joints of the hand.

FIG. 2 is a diagram of the exploded view of the CMC joint motion. The saddle-shaped joint motion can be seen as a multiple rotational axes joint.

FIG. 3 shows the front and back faces of an embodiment of the present invention.

FIG. 4 shows rotating side views of the implant of an embodiment of the present invention.

FIG. 5 shows a dimetric view of an embodiment of the present invention.

FIG. 6 shows a cross-section drawing of an embodiment of the present invention that passes through the center of the stem.

FIG. 7 shows a cross-section view passing off center through the implant.

FIG. 8 shows how an embodiment of the present invention rests in the bones of the hand.

FIG. 9 shows a palmar view of a right hand with a demonstrative implant installed.

FIG. 10 shows the front (left) and back (right) views of the artificial trapezium component of the implant.

FIG. 11 shows the top (left) and bottom (right) views of the artificial trapezium component of the implant.

FIG. 12 shows a dimetric view of the artificial trapezium design.

FIG. 13 shows non-limiting drawings of the left (left) and right (right) faces of the artificial trapezium component of the implant.

FIG. 14 shows front, side, and cross-sectional views of the first metacarpal stem design.

FIG. 15 shows front, side, and cross-sectional views of the first metacarpal stem design.

FIG. 16 shows front and cross-sectional views of the first metacarpal stem design with added threads.

FIG. 17 shows the embodiment of the present invention (modular) wherein the stem is a separate component from the artificial trapezium.

FIG. 18 shows the embodiment of the present invention (modular) wherein the stem slides onto the artificial trapezium from the front face.

FIG. 19 shows an embodiment of the present invention wherein the stem features an extruded cylindrical body with is concentrically inserted in a matching hole at the top of the artificial trapezium.

FIG. 20 shows the embodiment of the present invention wherein the stem features an extruded threaded base designed to thread onto the top of the artificial trapezium.

FIG. 21 shows the embodiment of the present invention wherein the stem features an extruded rectangular base.

FIG. 22 shows the embodiment of the present invention the stem features an extruded wedge/dovetail base.

FIG. 23 shows the embodiment of the present invention wherein the stem features an extruded angled curved body that maximizes the size of the stem while providing necessary curvature to match the first metacarpal shape.

FIG. 24 shows the embodiment of the present invention wherein the stem features an extruded circular base and rectangular extrusion with a hole.

FIG. 25 shows some of the tools potentially required for the installation of the present implant invention.

FIG. 26 shows the first step in the use of a first metacarpal drill guide with the present non-limiting implant.

FIG. 27 shows the second step in the use of the first metacarpal guide for use with the present non-limiting implant.

FIG. 28 shows a final step in the use of the first metacarpal guide for use with the present non-limiting implant.

FIG. 29 shows the first step in a potential surgical installation method with the non-limiting implant shown.

FIG. 30 shows the second step in a potential surgical installation method with the non-limiting implant shown.

FIG. 31 shows a third step in a potential surgical installation method with the non-limiting implant shown.

FIG. 32 shows a fifth step in a potential surgical installation method with the non-limiting implant shown.

FIG. 33 shows a sixth step in a potential surgical installation method with the non-limiting implant shown.

FIG. 34 shows a seventh step in a potential surgical installation method with the non-limiting implant shown.

FIG. 35 shows an eighth step in a potential surgical installation method with the non-limiting implant shown.

FIG. 36 shows a ninth step in a potential surgical installation method with the non-limiting implant shown.

FIG. 37 shows a tenth step in a potential surgical installation method with the non-limiting implant shown.

FIG. 38 shows another step in a potential surgical installation method with the present non-limiting design.

FIG. 39 shows an implant that was better optimized for manufacturing via basic machining processes.

FIG. 40 shows an implant having a side face that is configured to articulate with the normal shape of the trapezoid bone.

FIG. 41 shows an alternate embodiment wherein the stem and artificial trapezium are separate components.

FIG. 42 shows an implant stem component.

FIG. 43 shows the second component of a modular stem design.

FIG. 44 shows a final component of a modular stem design.

FIG. 45 shows an exploded assembly of a modular stem design.

FIG. 46 shows a diametric exploded view of a modular stem assembly.

FIG. 47 shows front, back, and side views of a modular stem completely assembled.

FIG. 48 shows the palmar surface of a hand skeleton with an installed non-limiting implant design.

FIG. 49 shows the dorsal surface of a hand skeleton with an installed non-limiting implant design.

FIG. 50 shows the posterior-anterior projection of an x-ray of a hand with an installed non-limiting implant design and transverse screw/pin.

FIG. 51 shows a size comparison of the implants used in experimental testing.

FIG. 52 shows the experimental testing setup of the thumb range of motion. Positional displacement.

FIG. 53 shows an anatomical coordinate system for a right hand.

FIG. 54 shows the experimental data table. Mean, standard deviation, and 95% confidence intervals were quantified for intact, fusion, fusion with partial resurrection, fusion with ligament transection, full trapeziectomy, and novel implant at all angles of circumduction

FIG. 55 shows a 3D view of the thumb's range of motion

FIG. 56 shows a xz view of the thumb's range of motion.

FIG. 57 shows a yz view of the thumb range of motion.

FIG. 58 shows a xy view of the thumb range of motion.

FIG. 59 shows the angular displacement of the thumb at each of the twelve angles. The angle between the first metacarpal and the second metacarpal as the thumb is passively loaded at each angle.

FIGS. 60A, 60B, 60C, 60D, 60E, 60F, 60G, and 60H shows how the stem locations on the top surface of the base may vary. Using the defined implant coordinate system representation of FIG. 61, the stem can move in the positive/negative x or y direction or a combination of both. FIG. 61 shows the varying angle of the stem to the base. α, θ, and φ are angles that determine the stem angle. The plane cuts the implant coordinate system representation into a positive half space where all angles exist. 0≤α≤180°, 0≤θ≤360°, 0≤φ≤180°.

FIG. 62 is an illustration of the fourth step in a potential surgical installation method with the non-limiting implant shown. A non-limiting temporary implant with a handle attached will be used to size the volume of the removed trapezium. The final size of the non-limiting implant will be chosen from the varying implant sizes in each surgical kit.

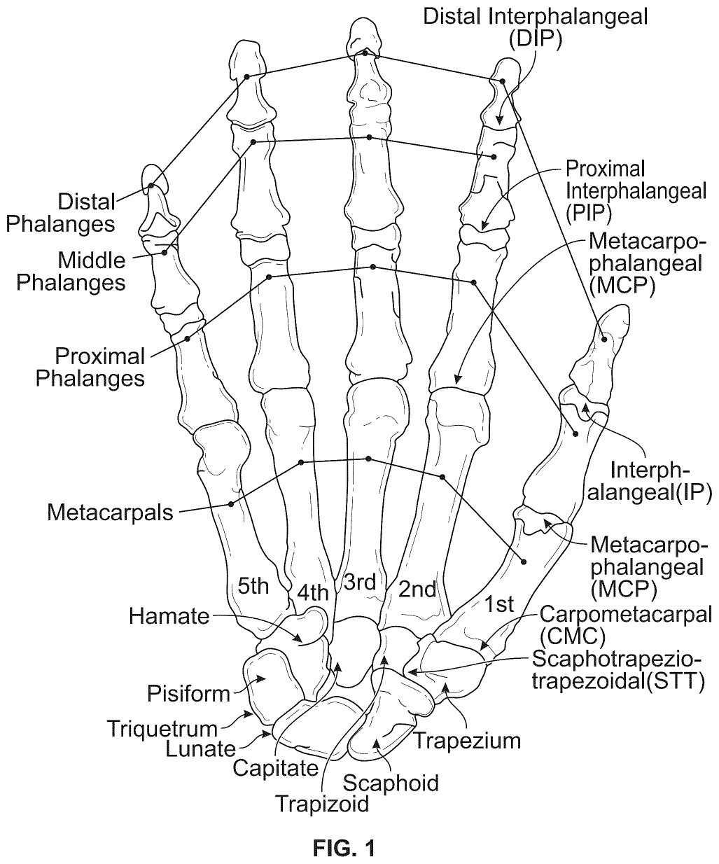

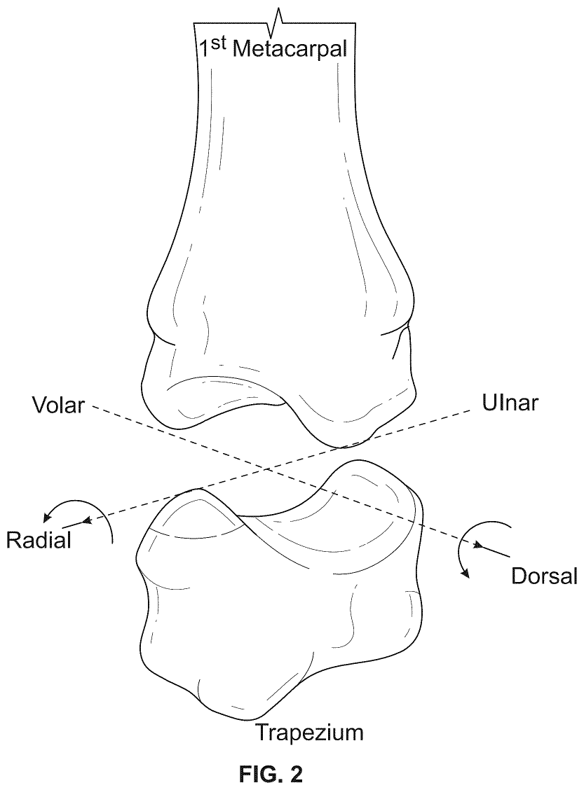

### DETAILED DESCRIPTION OF THE INVENTION

By making use of the following thorough description of the invention's preferred embodiment, the invention can be better appreciated. Detailed embodiments of the present invention are disclosed herein; however, it is to be understood that the disclosed embodiments are merely exemplary of the invention, which may be embodied in various forms, some of which may be significantly different from those in the disclosed embodiment, may be used to embodied processes, systems, and operational structures in accordance with the invention. Therefore, specific structural and functional details disclosed herein are not to be interpreted as limiting, but merely as a representative basis for teaching one skilled in the art to variously employ the present invention in virtually any appropriately detailed method, structure, or system. Further, the terms and phrases used herein are not intended to be limiting, but rather to provide an understandable description of the invention. However, they are considered to offer the best embodiment for disclosure reasons and to serve as a foundation for the claims made here, which specify the invention's realm of applicability.

A preferred embodiment of the present invention provides an implantable prosthetic that is a novel approach to surgically resolving CMC joint arthritis symptoms, using an implant to mimic a thumb CMC fusion. This implant is unique in that it resolves arthritic symptoms and maintains the patient thumb's range of motion and grip strength, while also avoiding constraint problems, a complicated surgical procedure, or a challenging recovery. These achievements are made through several unique design characteristics making it different from any other device on the market. The implant is also unique in that it can serve as both a primary treatment and/or a secondary treatment, available for both patients who have never had CMC surgery as well as patients who have had a failed CMC joint reconstruction. The present invention concerns an implantable prosthetic that effectively resolves thumb CMC arthritis pain by removing the arthritic trapezium carpal bone and replacing it with a stemmed, prosthetic trapezium, thus mimicking a thumb CMC fusion surgery The implantable prosthetic of the present invention replaces the trapezium bone with an artificial trapezium which has been designed as the central part of the implant. The artificial trapezium was designed in SolidWorks® CAD software using CT scans of real trapezium bones.

In one aspect of the present invention, using curvature-matching functions built into the software, the curvatures of the trapezium bone where the scaphoid and trapezoid bones articulate with the trapezium were replicated. The distal aspect of the trapezium implant was designed to fit the curvature of the base of the first metacarpal without the need for an osteotomy.

The implant was designed around the average dimensions of a female trapezium. CMC joint arthritis is much more common in females than males, hence the prototype was designed around the dimensions of an average female.

The final result is a small, easily manufactured trapezium replacement with maintained curvatures for the articulation to the scaphoid and trapezoid. Removal of the trapezium is important for pain relief as it eliminates bone-to-bone contact between arthritically damaged joints. Replacing the removed trapezium with a trapezium implant is an important part of preventing strength loss that results from the shortening of the thumb.

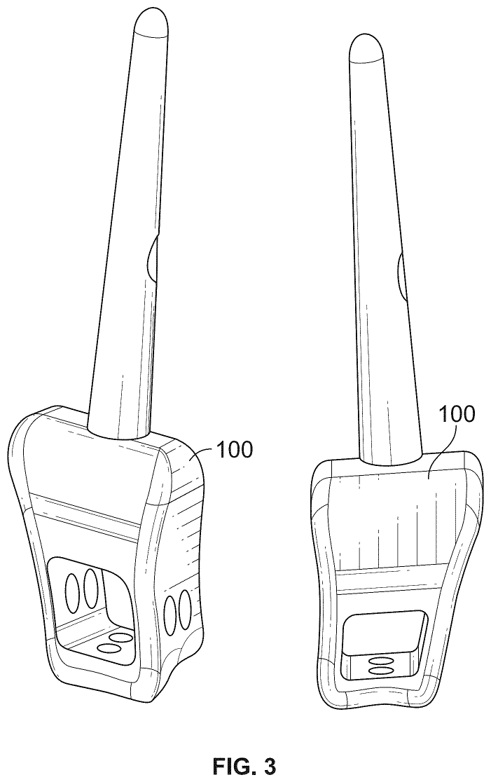

FIGS. 3-13 show a preferred embodiment of the present invention. FIG. 3 is a non-limiting drawing of the front and back faces of the implant design. This is an implant designed for use in the right hand. This image shows a complete assembly of this design, including key features such as the artificial trapezium 100 which acts as the central body, and the stem 110 which extends from it. Represented in the drawing is how conceptually the stem could extend perpendicularly from the artificial trapezium or at angles toward the sides of the implant.

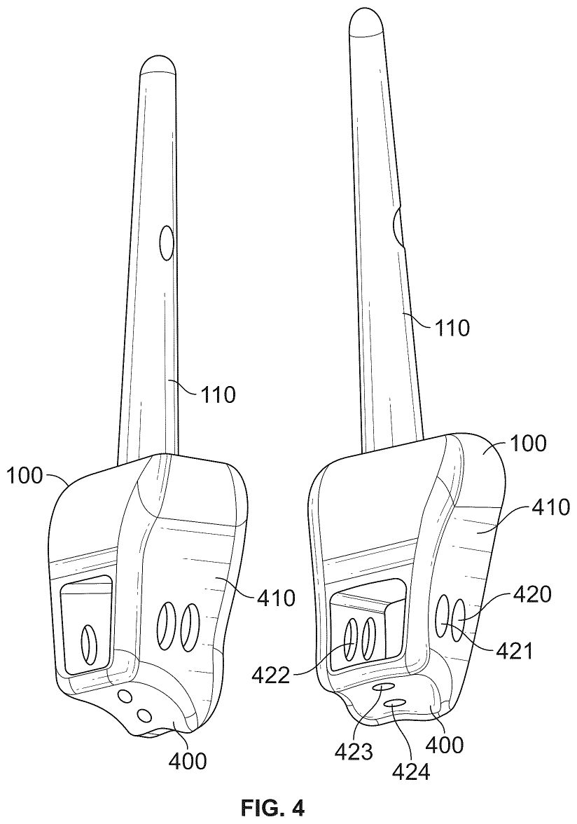

FIG. 4 shows non-limiting drawings of rotating side views of the implant in the design, showing the curvature features designed to articulate with the neighboring bones. The bottom curved face 400 is designed to articulate with the distal aspect of the scaphoid carpal bone, and the front face 410 is configured to articulate with the trapezoid bone. Holes in the implant to allow passage of sutures for ligament reconstruction, thus securing the implant in the trapezial space, are also shown in this figure.

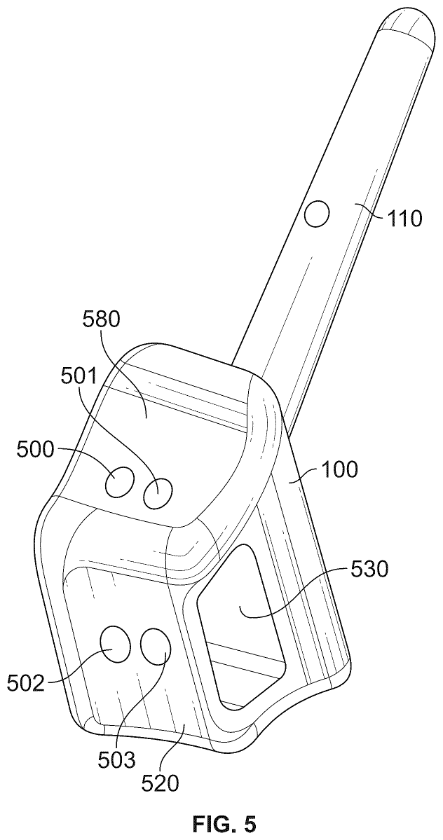

FIG. 5 shows a non-limiting dimetric view drawing of the design, providing further views of curvatures, holes, and windows used for fixation of the device in the body. As shown in FIG. 5, holes 500-503 are included in artificial trapezium implant 100, two at the trapezoid mating surface 580 (holes 500 and 501) and two at the scaphoid mating surface 520 (holes 502 and 503). To make room to allow for surgeons to pass these sutures, while also reducing the weight of the implant, implant 100 includes opening 530. Surface 580 has a curvature configured to match the opposing surface of the trapezoid.

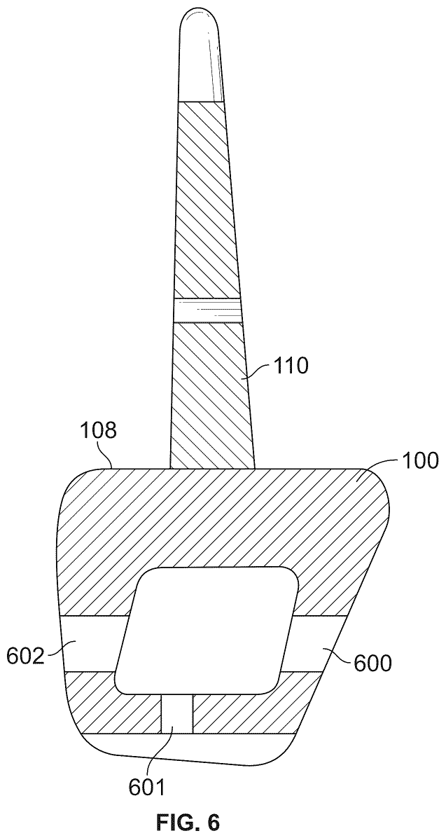

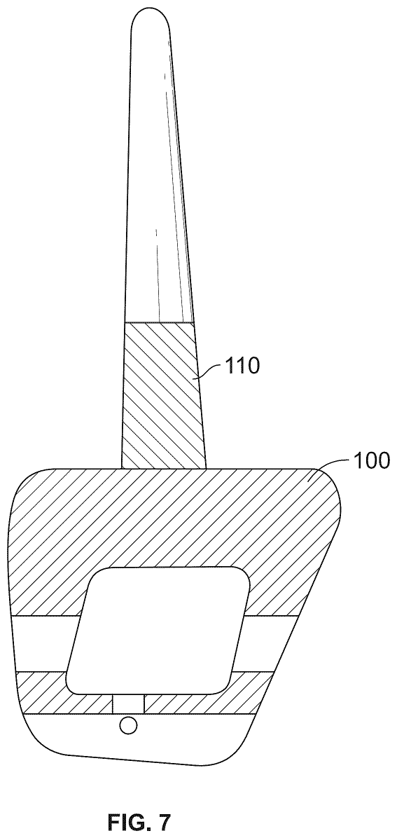

FIG. 6 shows a non-limiting cross-section drawing of the implant's side face. This cross-section passes through the center of the stem. The features demonstrated in this image include holes 600-602 through the front, back, bottom, and stem for suture fixation. FIG. 7 shows a separate non-limiting cross-section view drawing, this cross-section passing off center through the implant. FIG. 8 shows a conceptual non-limiting drawing of how the implant rests in the bones of the hand. This image shows a right hand with the average implant size installed in place of the trapezium bone. The stem is inserted in the first metacarpal approximately where a prototype iteration would rest. The image also demonstrates how the curvatures of the implant align with the trapezoid and scaphoid bones.

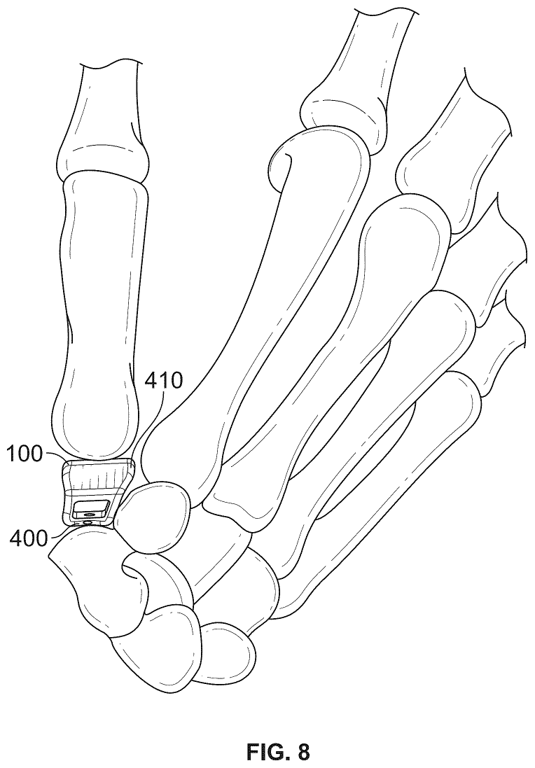

FIG. 9 shows a non-limiting illustrated palmar view of a right hand with a demonstrative implant installed in replacement of the trapezium bone. Here the placement of the implant within representative skin and tissue boundaries can be seen. An illustrative example of how the first metacarpal, second metacarpal, scaphoid, and trapezoid bones interact with the implant when the hand is at rest can be seen.

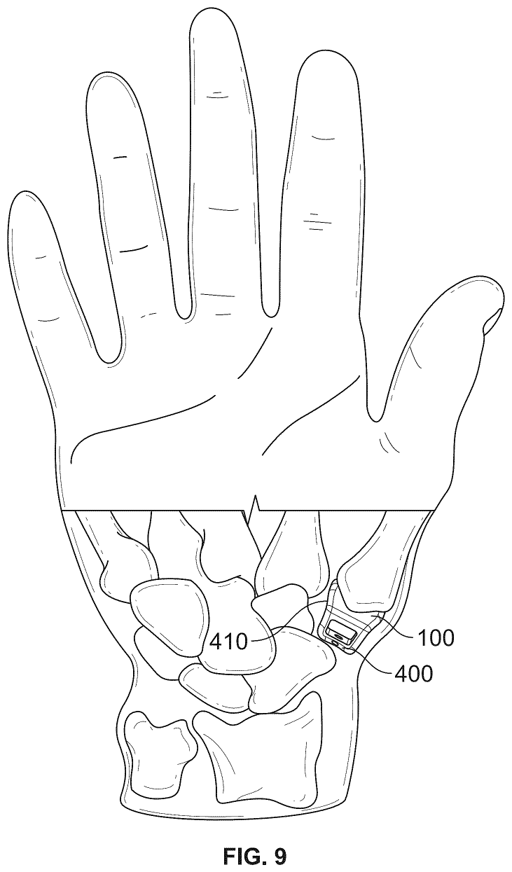

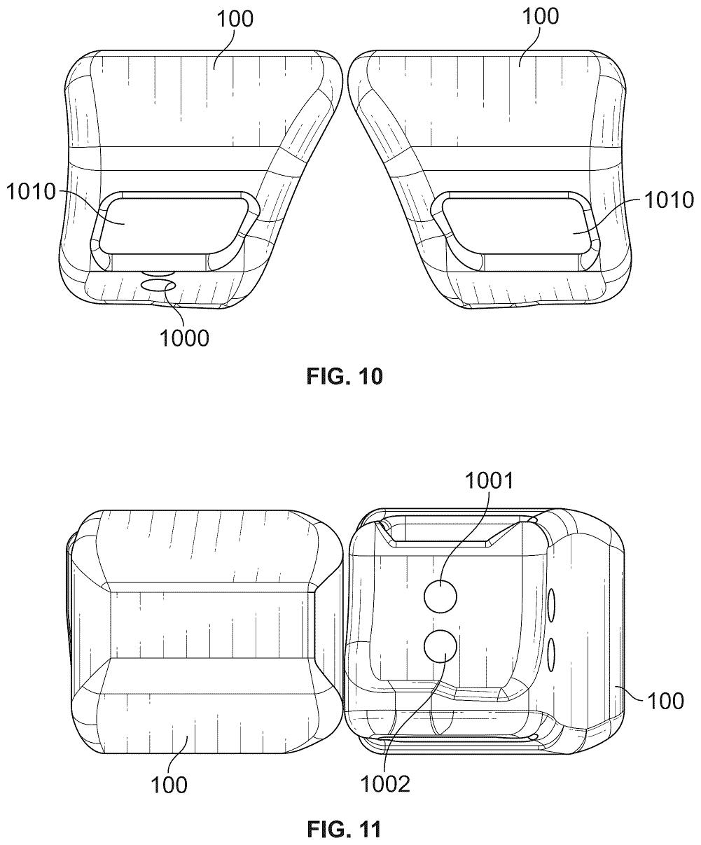

FIG. 10 shows the front (left) and back (right) view of non-limiting drawings of the artificial trapezium component of the implant. Detailed features such as curvatures, suture holes 1001-1002, and installation window 1010 are shown. FIG. 11 shows a top (left) and bottom (right) view non-limiting sketch of the artificial trapezium component of the implant. Features on the top are shown, such as curvatures or a flat surface from which the stem could protrude. Features shown on the bottom include suture holes, the shape of potential guide windows, and articulation curvatures are shown.

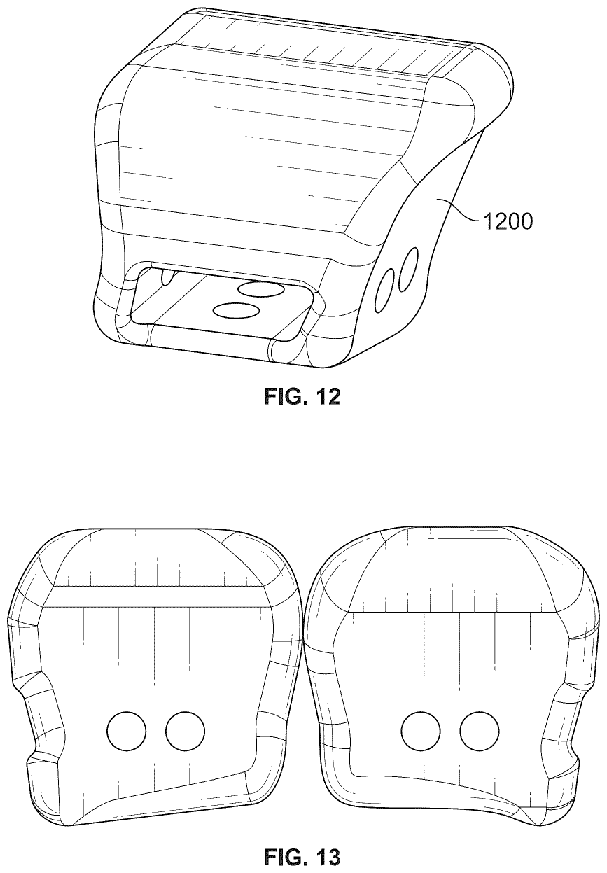

FIG. 12 shows a non-limiting dimetric view drawing of the artificial trapezium design. In this view, a conceptual illustration of what views a surgeon may have while fixing and installing the implant component is shown. On the right-hand side of the image is surface 1200 intended for articulation with the neighboring trapezoid bone. FIG. 13 shows non-limiting drawings of the left (left) and right (right) faces of the artificial trapezium component of the implant. The right-hand face is intended for direct contact with the neighboring trapezoid bone. The left-hand face would point out of the incision during installation. Both faces feature holes for fixation with sutures.

What makes this implantable trapezium prosthetic unique is the design and implementation at the CMC joint. Another technology maintains a curved saddle for the top of the artificial trapezium, allowing the first metacarpal to articulate on the top of the implant. The implantable trapezium prosthetic of the present invention is also configured to sit in the saddle at the base of the first metacarpal (i.e. there is convexity to the distal aspect of the implant that is configured to match the concavity at the base of the first metacarpal) however the joint is constrained by the placement of a stem. This convexity can be seen in FIG. 13.

Additionally, it is important to have access to a collection of prostheses in various sizes to accommodate diverse anatomies should a prosthesis be necessary. As a result, based on anatomical observation, one particular form of the present invention provides prostheses in at least three sizes, from the smallest to the largest. The artificial trapezium implant may be of at least three sizes based on average trapezium bone sizes ranging from small, medium, and large. Trapezium bone sizes vary enough between patients of differing sizes and gender that providing multiple trapezial implant sizes will be critical for achieving the best patient outcomes. A one-size-fits-all approach could result in discomfort and lack of optimal function, as good motion and grip strength are achieved by maintaining the patient thumb length.

As the number of different sizes of a pair of prostheses to be offered in the set can vary, the inclusion of only three prostheses (a pair) in a set is not meant to be restrictive. It is preferred, though, to have three to five different-sized prostheses (a pair) in a set available as needed. However, without going outside the bounds of the scope, more or fewer prostheses (a pair) can be offered in a set.

If desired, a set of trial prostheses (a pair, not shown) that typically match the prosthesis' proportions may also be given. The final prosthesis (a pair) to be implanted can be chosen with the aid of these trial prostheses (a pair). If desired, the surgical set may also include other tools including drill bits, drill guides, etc. (not pictured).

A titanium plasma spray coating may be applied to any or all of the surfaces of the prostheses, which are made of highly polished 6Al4V titanium, 314 stainless steel, and/or cobalt chromium in one preferred embodiment of the invention. Other materials include titanium, stainless steel (SST), aluminum, polyetheretherketone (PEEK), cobalt chrome, polyethylene, polycarbonate, polyurethane, nylon, carbon fiber, other biocompatible metals and alloys, other biocompatible polymers. This is meant for long-term biological fixation. If offered, the trial prostheses (a pair) may be constructed from Al360/Al360 aluminum or another biocompatible material.

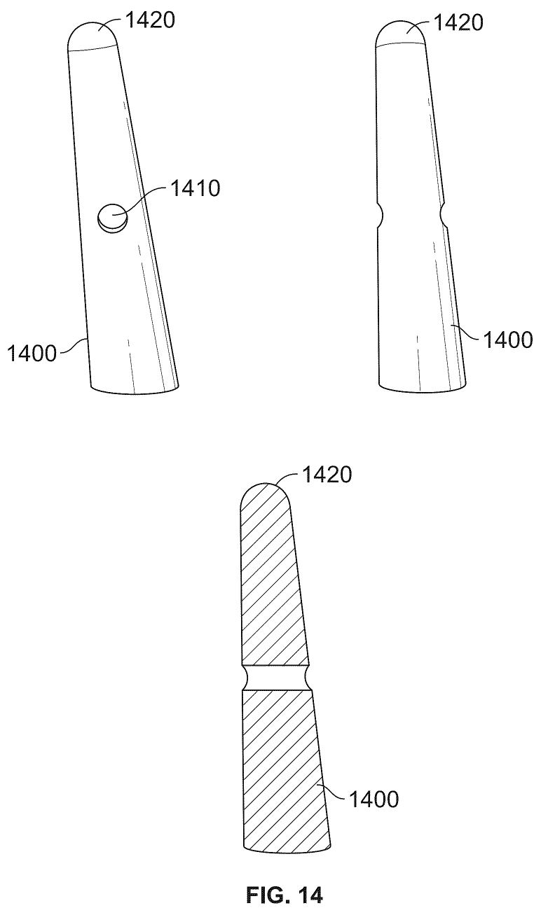

The first metacarpal stem implant is used to allow the implantable trapezium prosthetic to mimic thumb CMC fusion, thus eliminating motion at the thumb CMC saddle joint. FIG. 14 shows front, side, and cross-sectional drawing views of a non-limiting first metacarpal stem design 1400. The stem is intended for direct insertion into a hole in line with the center axis of the intramedullary canal of the first metacarpal. In this design, the stem is frusto-conical to account for changing diameters of the first metacarpal. A transverse hole 1410 through the side of the stem is shown which could be used for the insertion of a pin or screw which would prevent the stem from rotating in the first metacarpal after insertion. A rounded tip of 1420 is used to allow full contact of the stem with the end of the hole in the first metacarpal and for patient comfort.

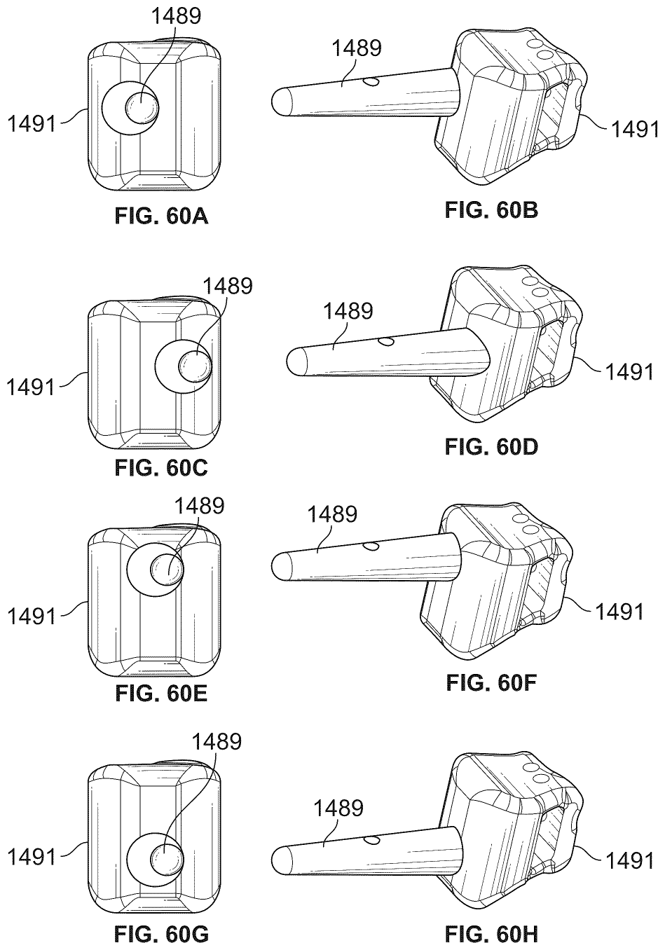

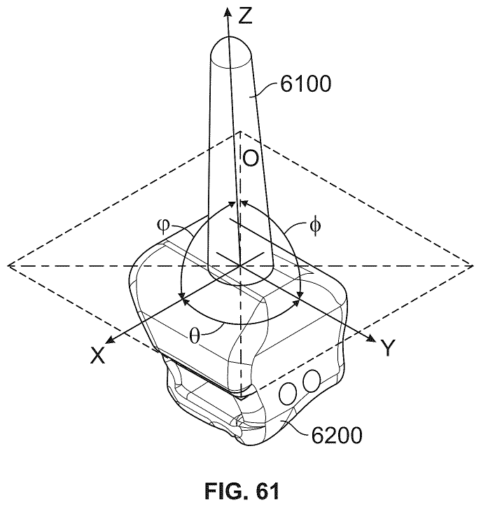

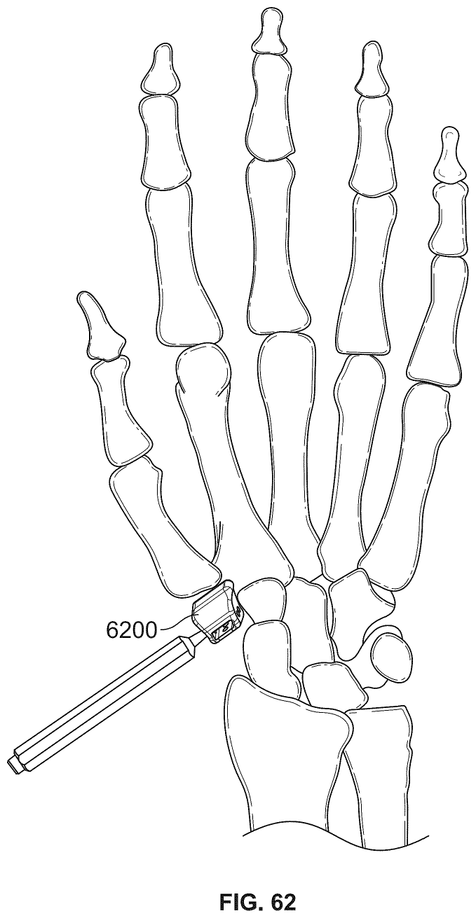

In other embodiments of the present invention, as shown in FIGS. 60A-60H, stem 1489 does not need to sit centered on the base but can be placed anywhere such as having a dorsal offset on base 1491 to more accurately align with the center axis of the intramedullary canal of the of the first metacarpal. In a preferred embodiment, stem 1489 is adapted to be positionable on base 1491. In other embodiments, a kit of multiple bases is provided with static stems in various positions on the base for selection by a surgeon. The convex distal aspect of the trapezial component is configured to sit in the concavity at the proximal aspect of the first metacarpal, with the stem configured to align with the center axis of the intramedullary canal.

As shown in FIG. 61, stem 6100 may have an angle that can vary in any direction. The stem is always placed in line with the central axis of the thumb, though by angling the stem, the thumb metacarpal position relative to the trapezium can be adjusted. In a preferred embodiment, stem 6100 is adapted to be angularly positionable on base 6200. In other embodiments, a kit of multiple bases is provided with stems at various angles for selection by a surgeon.

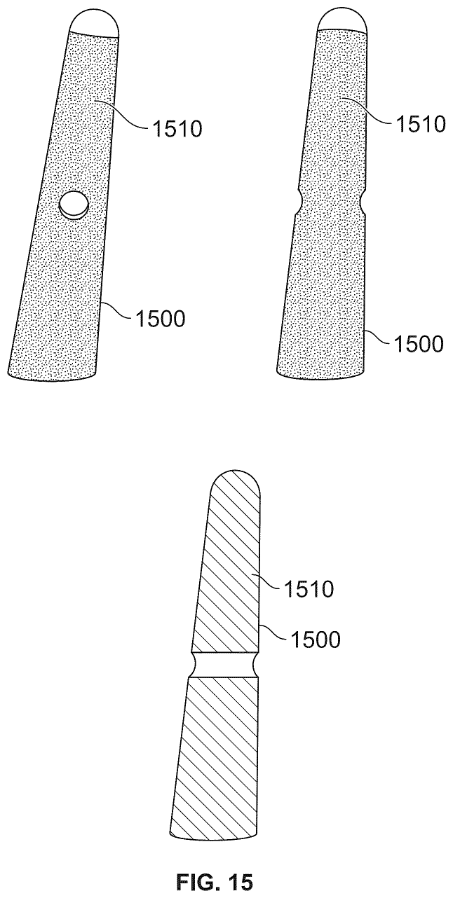

FIG. 15 shows front, side, and cross-sectional drawing views of a non-limiting first metacarpal stem design 1500. In this design, texture, porous, or titanium plasma spray 1510 is illustrated on the stem. Rough texture, porous, or titanium plasma spray can be applied to the stem to promote bone on-growth and assist in mechanical and/or biological fixation.

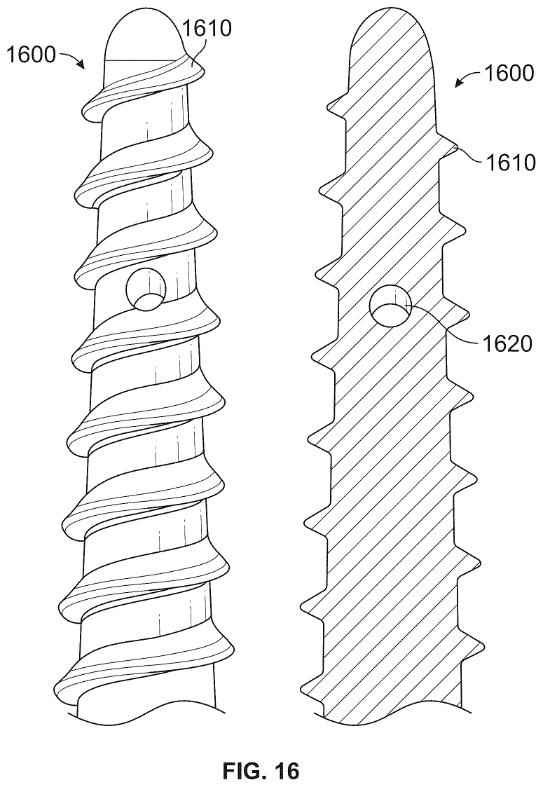

FIG. 16 shows front and cross-sectional views of a non-limiting first metacarpal stem design 1600 with added threads 1610. In this design, the same frusto-conical stem previously illustrated has threads added for rigid insertion into the first metacarpal. A hole 1620 is included for pinning in the first metacarpal to prevent the screw stem from backing out of the hole in the first metacarpal with extensive thumb use.

As with the trapezium implant, the stem dimensions were based on the average dimensions of a female first metacarpal. Multiple stem sizes may be available for variable spacing and variable patient hand sizes. In present designs, the stem may either extend from the artificial trapezium perpendicularly or at an angle. The angled stem is configured to mimic the ideal position of thumb CMC fusion which involves positioning the thumb in both extension and abduction (away from the palm). Angling the stem allows the thumb to sit at a more natural position when the hand is at rest. When attached to the artificial trapezium and inserted in the patient's first metacarpal, the stem centerline continues to match the centerline of the first metacarpal, thus shifting the first metacarpal relative to the trapezium. This is also typically done in thumb CMC fusion surgery.

In the present invention, the stem is frusto-conical, tapering with a (3%) grade from a wide base to a narrow tip, though non-tapering stems could also be utilized. The cone shape is designed to accommodate the narrowing of the midportion of the first metacarpal. Alternately, the stem may be non-frusto-conical as well, configured to match the normal shape of the first metacarpal intramedullary canal, which would prevent rotation without a transverse screw. A non-frusto-conical stem should be as broad as possible so as to fill the intramedullary space without removing the cortical portion of the first metacarpal, as a thicker stem will have greater strength and rotational control. The thick base of the first metacarpal allows for a thicker stem, but the stem must taper as it goes into the first metacarpal to account for the decreasing diameter of the bone.

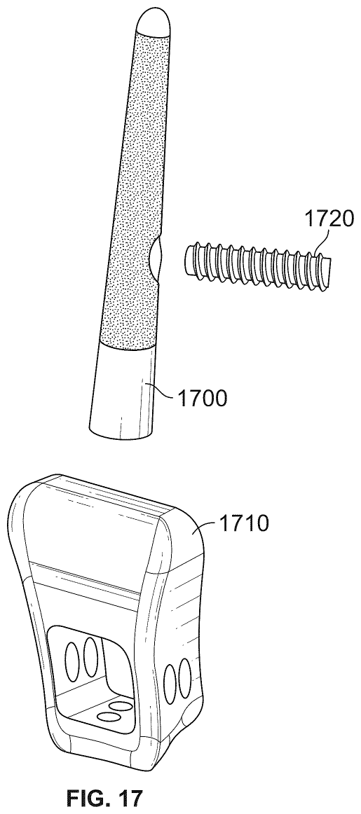

The combination of the stem and artificial trapezium comprises the complete implant. The stem and trapezium components could be left as one piece, creating a mono-block implant. However, the present design also considers having a modular implant consisting of separate stem and artificial trapezium components for ease of surgical installation and minimizing the surgical window. This design has the stem inserted in the patient's first metacarpal before attaching mechanically to the artificial trapezium. For example, FIG. 17 shows a non-limiting illustration of the design in an exploded view. Here, stem 1700 is a separate component from the artificial trapezium 1710. A non-specified mechanical connection can be used to attach the stem to the artificial trapezium. A threaded pin 1720 is included in the image for locking the stem in the first metacarpal. Alternatively, to this exploded view, the implant can be manufactured and used as one solid piece.

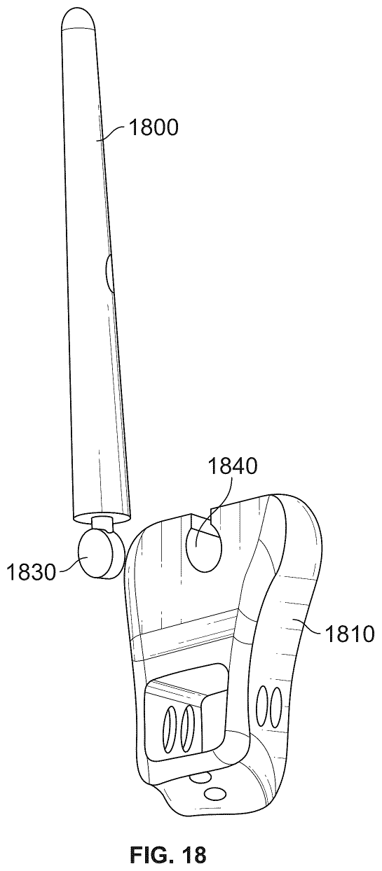

FIG. 18 shows a non-limiting conceptual drawing for mechanically attaching stem 1800 to the artificial trapezium 1810 to assemble the full implant. Here stem 1800 includes a key 1830 that slides into the artificial trapezium from the front face through keyhole 1840. The stem could then be fixed in this position using a locking screw in the same hole the stem slides into.

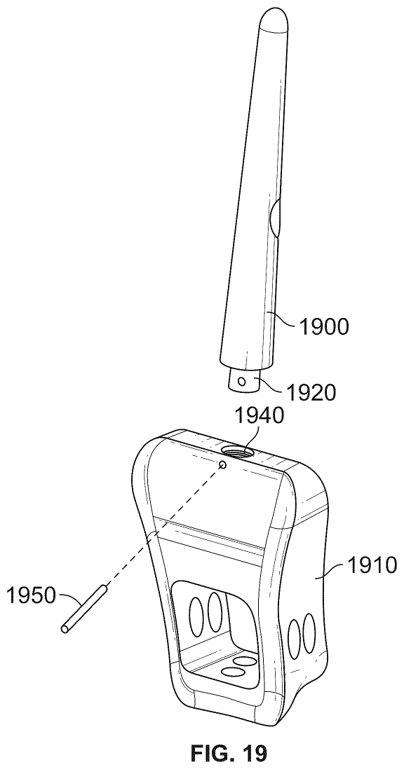

FIG. 19 shows a non-limiting conceptual drawing for mechanically attaching the stem 1900 to the artificial trapezium 1910 to assemble the full implant. In this design the stem features an extruded cylindrical body 1920 with is concentrically inserted in a matching hole 1940 at the top of the artificial trapezium. A screw or pin 1950 can then be inserted through matching through holes in the stem and artificial trapezium to lock the components together.

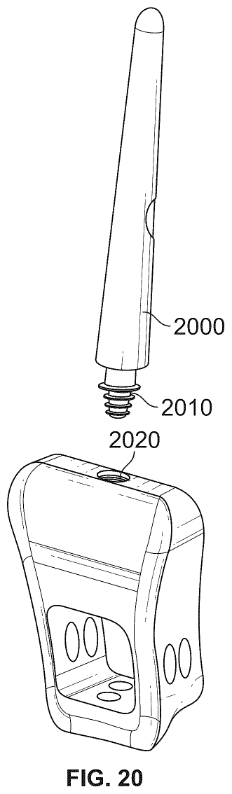

FIG. 20 shows a non-limiting conceptual drawing for mechanically attaching the stem to the artificial trapezium to assemble the full implant. In this design, the stem 2000 features an extruded threaded base 2010 designed to thread onto the top of the artificial trapezium 2020. Fixation of the stem with a pin or screw and fixation of the artificial trapezium with sutures would prevent the components from disengaging after implantation. This implant is designed for press-fit with the use of a transverse screw or pin, non-cemented use.

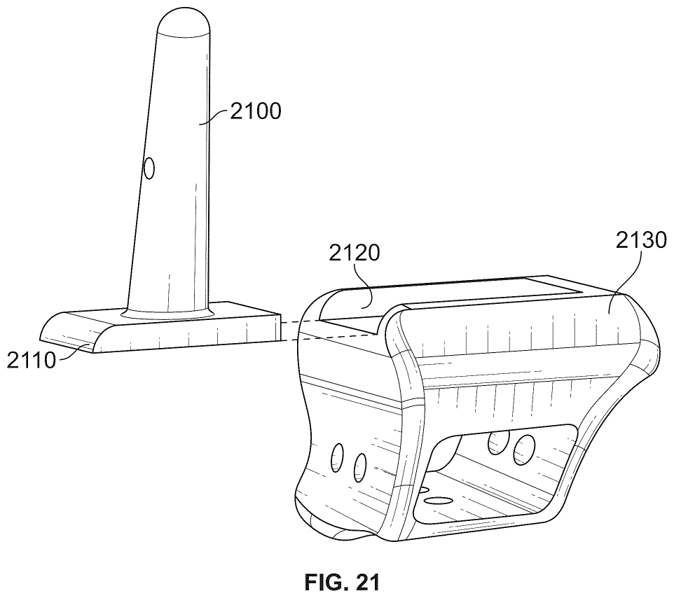

FIG. 21 shows a non-limiting conceptual illustration for mechanically attaching the stem to the artificial trapezium to assemble the full implant. In this design, the stem 2100 features an extruded rectangular base 2110. Here the stem with a rectangular base slides into the slot 2120 on the artificial trapezium 2130. As the stem would be implanted first, the trapezial component would be slid onto the stem after the stem was secured to the first metacarpal.

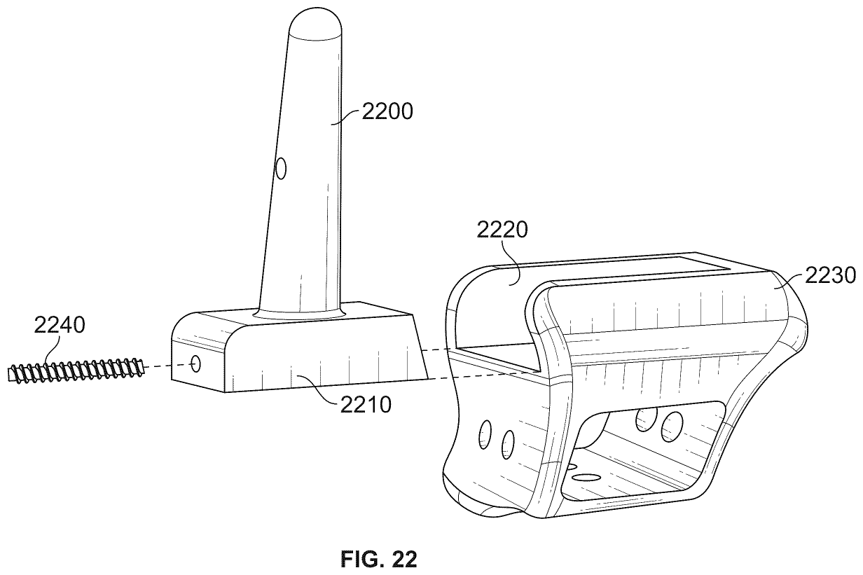

FIG. 22 shows a non-limiting conceptual illustration for mechanically attaching the stem to the artificial trapezium to assemble the full implant. In this design, the stem 2200 features an extruded wedge/dovetail base 2210. Here the stem with wedge/dovetail base slides into the slot 2220 on the artificial trapezium 2230. A screw or pin 2240 can then be inserted through matching through holes in the stem and artificial trapezium to lock the components together.

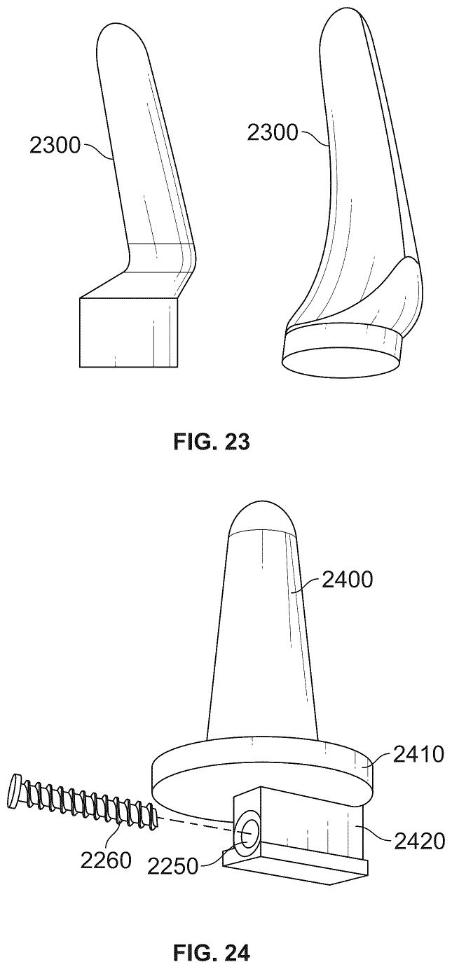

FIG. 23 shows a non-limiting conceptual illustration for the stem part of the full implant assembly. In this design, the stem 2300 features an extruded angled curved body that maximizes the size of the stem while providing the necessary curvature to match the first metacarpal. FIG. 23 also highlights how the stem is configured to match the offset of the base to the central axis of the first metacarpal.

FIG. 24 shows a non-limiting conceptual illustration for the stem part of the full implant assembly. In this design, the stem 2400 features an extruded circular base 2410 and rectangular extrusion 2240 with hole 2250. The rectangular extrusion slides into a slot on the artificial trapezium. A screw or pin 2260 can then be inserted through matching through holes in the stem and artificial trapezium to lock the components together.

This implantable prosthetic is designed so that the original trapezium is removed, a hole is drilled in the first metacarpal base, the stem is inserted into the first metacarpal hole, and the artificial trapezium is attached to the stem and inserted in the place of the native trapezium. By joining the artificial trapezium to the stem, articulation between the first metacarpal and trapezium is eliminated and a fixed, i.e. “fused” joint is created. In this way, the device effectively mimics thumb CMC fusion surgery, without the need to fuse bones or the difficult recovery that follows. By constraining this joint, the device should not fail after continued use in the way unconstrained devices on the market often fail. In addition, the use of a stem implant and artificial trapezium combination allows for the thumb length and ligament tension to be maintained. The combination of these unique design features, as well as the engineered curvatures for the scaphotrapeziotrapezoidal (STT) joint, results in an implant that maintains both grip strength and range of motion for arthritic patients.

For further constraint, the implant is fixed at the first metacarpal and sutured to the trapezoid and scaphoid, preventing dislocation or other complications while healing. To achieve this, the first metacarpal implant has a transverse through-hole about halfway up the length of the stem, allowing a bone pin, screw, or other pinning/locking method to be inserted.

To fix the stem transversally, a matching hole must be drilled in the first metacarpal. This will be done using a guide jig, provided with the implantable trapezium prosthetic, which will allow surgeons to easily locate the precise location to drill. The drill guide is designed to attach to the side of the implant after it has been installed in the hand, aligning and locking into features of the artificial trapezium. When locked in place, a drill guide external to the thumb will be in line with the through-hole in the stem. This will allow a surgeon to insert a drill through the drill guide, external from the thumb, and drill through soft tissue and bone. This will create a transverse hole through the bone, concentric and identical in size to the transverse hole in the stem implant. A pin or screw can then be inserted through the first metacarpal and stem implant to fix the stem in place. The use of rough texture, porous, or titanium plasma spray surfaces on the stem in combination with the transverse hole will create a sturdy mechanical and biological fixation in the first metacarpal unlike any other product on the market. This implant is designed for press-fit with the use of a transverse screw or pin, non-cemented use.

Sutures and suture anchors will be used to fix the artificial trapezium component to the scaphoid and trapezoid bones. The artificial trapezium includes many features to assist in this fixation. Holes are included on the artificial trapezium implant, at least two at the scaphoid mating surface and two at the trapezoid mating surface. Slots are also included on the surfaces of the trapezium implant which allow sutures to pass or be wrapped. To allow easy access for surgeons to pass sutures, while also reducing the weight of the implant, large openings are machined out of the body of the artificial trapezium, providing a window for suturing.

Sutures and suture anchors are not included with the implantable trapezium prosthetic as these are readily available in many types, materials, and sizes from multiple sources. In addition, the implant is not designed for one specific fixation method but instead for many. Though a specific procedure has been designed and will be recommended, surgeons can use the slots, holes, and windows to fix the device in a variety of different ways. The implant is designed for numerous combinations of fixation techniques that will allow surgeons to install the device with versatility and preference or to adapt to unexpected differences between patients. It is recommended that the implant be fixed to the neighboring trapezoid and scaphoid carpal bones with suture anchors. Here, surgeons would insert the anchor of their choice into the scaphoid and trapezoid bones and pass sutures between the bones and artificial trapezium using the suture holes. This would recreate the normal ligamentous attachments of these bones to the native trapezium. As the trapezium is removed, these ligaments are released, thus requiring reconstruction to secure the implant in place.

These fixation designs are both important and unique to this device. Suture anchors have been used in the past to fix implants in place. However, both the first metacarpal stem for this application and the use of screws or pins for fixing the stem is unique. A potential source of failure with the stem implant to the first metacarpal is the failure of bone on growth. By fixing the stem in the first metacarpal with a rough texture, porous, or titanium plasma spray surface, bone on-growth should be more easily facilitated as the stem will not be able to slide or rotate, damaging on-growth progress. Anchoring the artificial trapezium to the trapezoid and scaphoid will allow for a better constraint of the implant, allowing patients to use their hands for high-strength applications.

Another unique aspect of this design that will benefit both patient strength and surgical implantation is the use of variable sizes, lengths, and spacers. Maintaining thumb length and ligament tension is critical for maintaining grip strength. To allow surgeons the ability to further fine-tune these key aspects of the thumb while inserting the implant, variable stem sizes or spacers can be used to create incrementally larger gaps between the first metacarpal and artificial trapezium. This will allow surgeons to maintain tension in the thumb ligaments and replicate the original thumb length, improving patient outcomes post-op. Varying sizes of implant components will also allow surgeons to account for varied hand sizes from one patient to the next. The use of separate stem and artificial trapezium components, with a mechanical connection between the two, also means a spacer could be used. A spacer matching the mechanical connection of the stem and trapezium could be inserted in between the two to further lengthen the implant. For example, one design uses threaded male and female connections between the stem and artificial trapezium. Spacers with matching threads are available for insertion between the stem and artificial trapezium in this design. The bottom of the spacer will attach to the top of the artificial trapezium, and the stem will attach on top of the spacer. This design also allows multiple spacers to be stacked and threaded on top of one another. Alternatively, the device may be offered with multiple first metacarpal stem implants of varying lengths to achieve the same result. This allows the surgeon to adjust the position of the stem and trapezial component for optimal resting position before locking/fixing the stem and artificial trapezium.

Constraint mechanisms used in this design allow for the implantable trapezium prosthetic in current non-limiting designs to be made of solid metal, keeping the implant simple and the joints comfortable for the patient. The entire non-limiting design device is constructed of (just two pieces of) metal: the stem and the artificial trapezium. Existing thumb implant devices necessitate complicated fixation such as being adhered to the surfaces of bones. This requires manufacturing with adhesion-compatible materials with lower strength than metal, such as polyethylene. Constrain mechanisms in this presented non-limiting implant design do not require adhesive, meaning the entire implant can be made of two simple, solid pieces of metal. This allows for several benefits, including increased strength of the device, easy production and machining, and increased comfort for the patient as bone-to-metal joins are known to be comfortable for articulation. The simple design of this implant and quick mechanical connection between components for assembly means installation will be fast and simple for surgeons.

### Surgical Installation Procedure

The following surgical installation procedure pertains to the use of the described non-limiting two-component design in which a frusto-conical stem and artificial trapezium are separate components with a mechanical connection. The procedure also pertains to the use of a hypothetical complete product, which would be delivered as a surgical kit. This kit would include the full implant in necessary varying sizes, as well as all tools that are unique to or necessary for this procedure. To begin, a dorsal incision is made over the scaphotrapeziotrapezoid and thumb carpometacarpal joints. The branches of the superficial branch of the radial nerve, as well as the dorsal branch of the radial artery, are protected. A longitudinal arthrotomy is made over the dorsal aspect of the thumb carpometacarpal joint and trapezium. The soft tissue attachments to the trapezium are then sharply released. The trapezium is removed, either en bloc or in a piecemeal fashion. Here, a potentially included carpal bone removal corkscrew may be used for extraction of the trapezium.

Preserving the remaining articular surface, a drill is used to create a hole in the dorsal one-third of the first metacarpal articular surface, entering the intramedullary canal of the first metacarpal. Osteophytes may be debrided through the subchondral bone of the first metacarpal base is preserved. Exact matching of hole diameter to implant stem diameter is achieved using the included drill bits. For frusto-conical stems, a hole may be pre-drilled and an included frusto-conical drill/awl is then used to create the proper shape and space for the stem. The implant stem is then inserted loosely. At this point, patient trapezium size should be noted and the included various-sized trapezial implants should be tested for the best fit in the patient. The appropriate artificial trapezial implant is then inserted in the hand and connected to the stem. An included guide jig is then used to drill and place an interlocking screw or pin through the first metacarpal and the transverse hole in the stem of the implant.

To anchor the trapezial component of the implant, two suture anchors are placed, one in the trapezoid and one in the scaphoid. These can either be placed dorsally or on the articulating surfaces. Sutures from the anchors are then passed through the trapezial component of the implant utilizing features such as windows, suture holes, and grooves. Sutures are then tied securely over the radial aspect of the implant. The implant must be properly reduced when tying the sutures. The capsule is then repaired over the implant. Finally, the subcutaneous tissue and skin can be closed in a standard fashion.

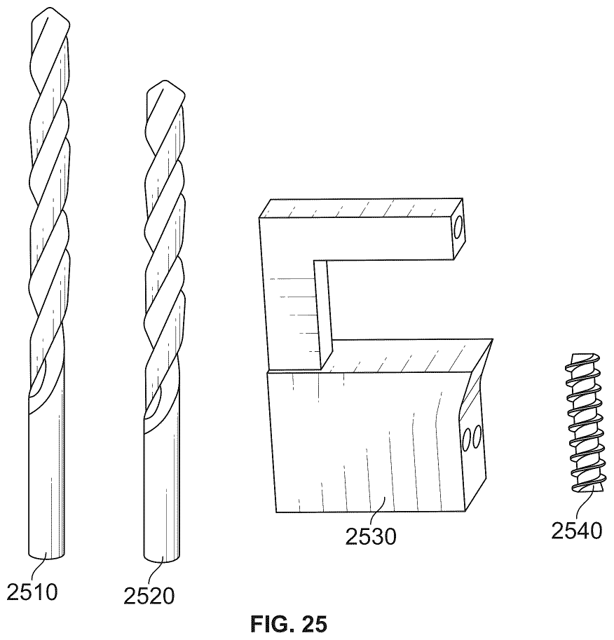

FIGS. 25-38 show other installation procedures. For example, FIG. 25 shows some of the tools potentially required for the installation of the present implant invention. These tools include a drill bit 2500 for precise drilling of a stem insertion hole in the first metacarpal, a drill bit 2510 for creating a transverse hole in the first metacarpal, a drill guide 2520 for aligning the hole in the first metacarpal, and a threaded pin 2530 for fixing the stem to the first metacarpal.

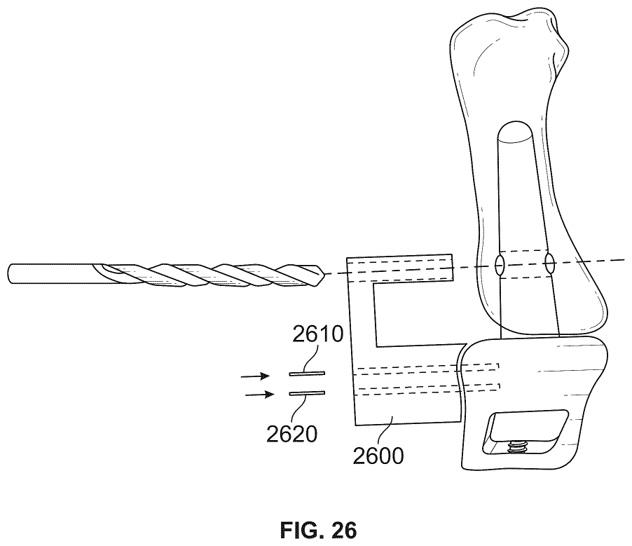

FIG. 26 shows a drawing of the first step in the use of a first metacarpal drill guide 2600 with the present non-limiting implant. This guide is designed to allow surgeons to percutaneously drill a hole through the first metacarpal that aligns with the hole in the implant stem. The guide is designed to align with two of the suture holes on the back face of the artificial trapezium. Pins 2610 and 2620 can be inserted through the matching holes to align the guide with the implant. When the guide is aligned with the holes on the artificial trapezium, a third hole at the top of the guide is in line with the transverse hole in the stem. A drill bit of matching diameter can be inserted through the drill guide to make a percutaneous hole through the first metacarpal of the thumb.

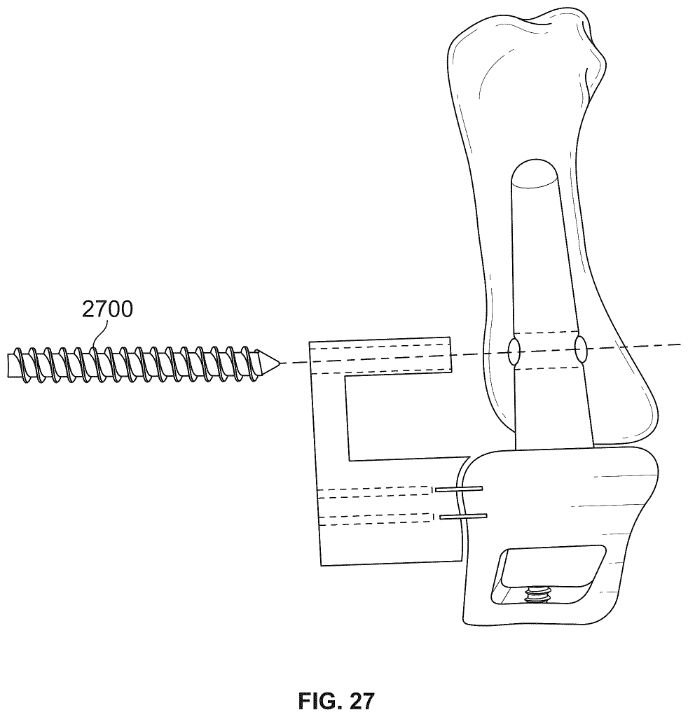

FIG. 27 shows a drawing of the second step in the use of the first metacarpal guide for use with the present non-limiting implant. After the matching concentric hole has been drilled through the first metacarpal, a pinning mechanism such as the illustrated threaded pin 2700 can be inserted to lock the stem in the first metacarpal.

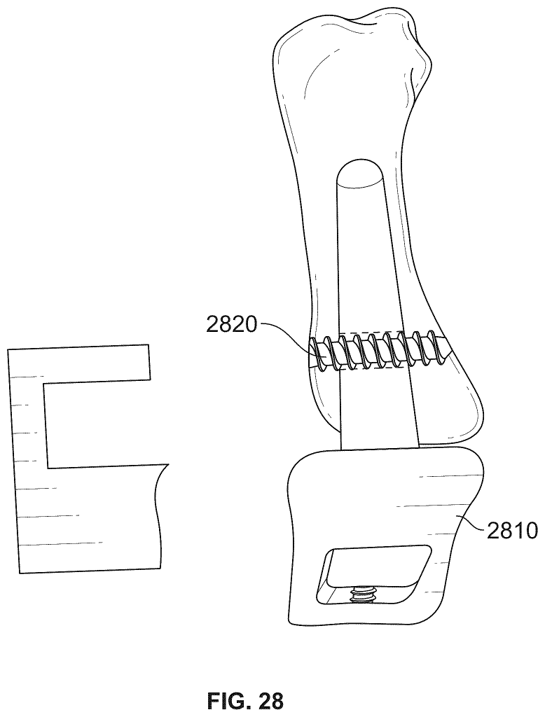

FIG. 28 shows a drawing of the final step in the use of the first metacarpal guide for use with the present non-limiting implant. To complete the fixation of the stem, remove the guide 2800 from the implant 2810 with the pin or screw 2820 in place. The stem will be locked in place by the transverse pin, preventing rotation or sliding.

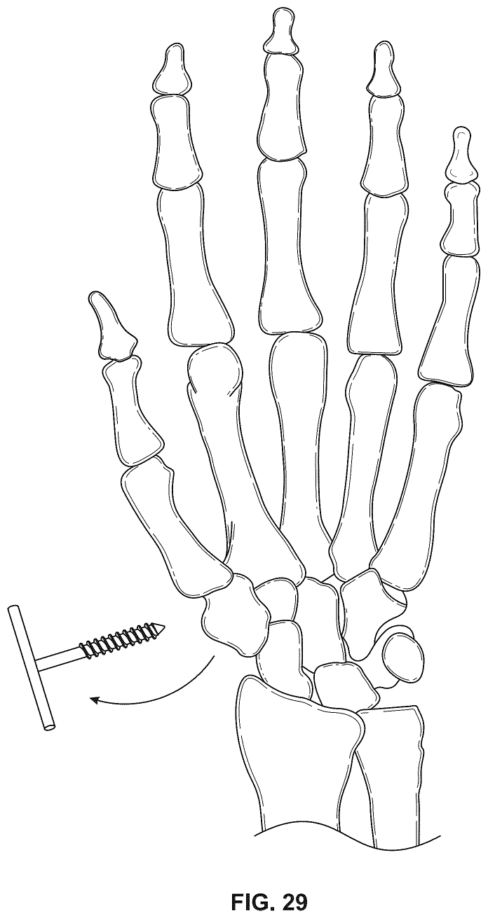

FIG. 29 shows an illustration of the first step in a potential surgical installation method with the non-limiting implant shown. The procedure is performed on the right hand. Here, the first step is to remove the trapezium bone from the hand. This can be done using a carpal removal screw as illustrated.

FIG. 30 shows an illustration of the second step in a potential surgical installation method with the non-limiting implant shown. The provided drill bit 3000 matching the cylindrical, frusto-conical, or potential another shape of the stem is used to drill a hole 3010 through the center line of the first metacarpal 3020, starting at the base.

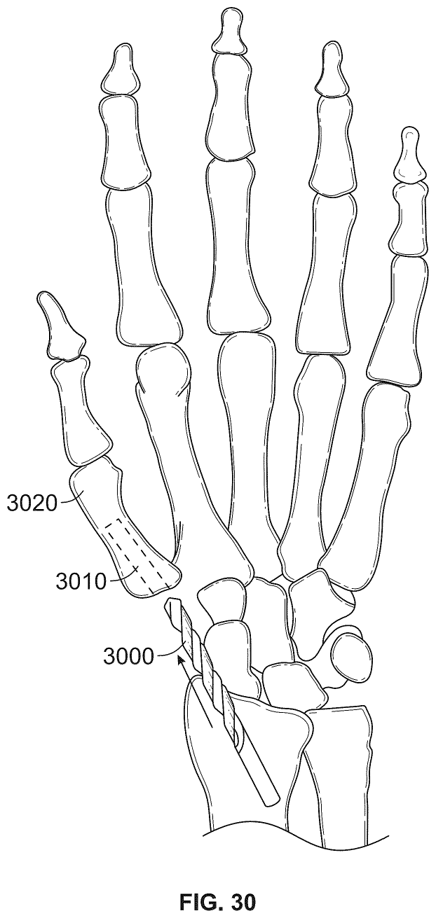

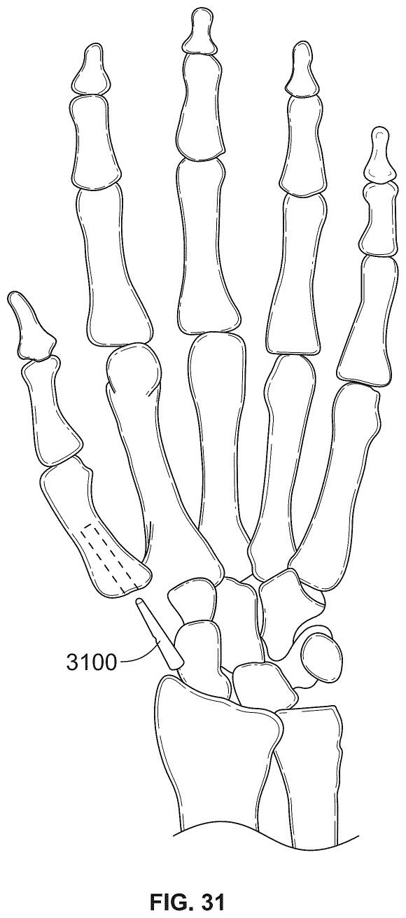

FIG. 31 shows an illustration of the third step in a potential surgical installation method with the non-limiting implant shown. This step of the procedure pertains to a design in which the stem and artificial trapezium components are separate. The third step in this procedure is to insert the stem 3100 of the implant in the first metacarpal hole.

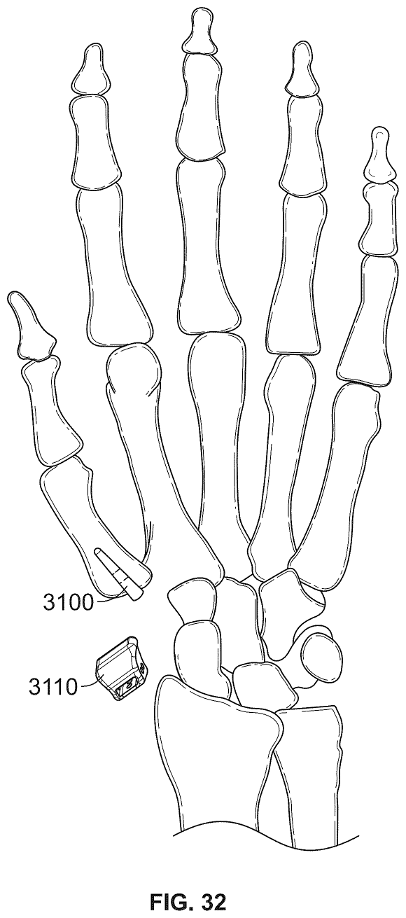

FIG. 32 shows an illustration of the fifth step in a potential surgical installation method with the non-limiting implant shown. This step of the procedure pertains to a design in which the stem 3100 and artificial trapezium 3110 components are separate. The artificial trapezium is to be inserted in the gap left by removing the trapezium from the patient. Using a mechanical locking mechanism such as the previously described methods, the stem is locked to the artificial trapezium. Upon removal of the trapezium, as shown in FIG. 62, an additional potential surgical installation method is to estimate the size of the implant base using temporary varying-sized bases.

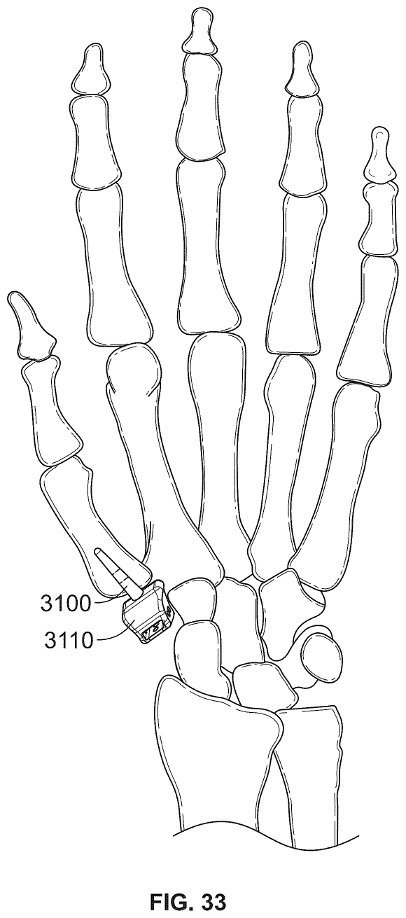

FIG. 33 shows an illustration of the sixth step in a potential surgical installation method with the non-limiting implant shown. Here, the complete stem and artificial trapezium combination have been installed in the hand. The surgeon should properly align the curvatures of the artificial trapezium and the stem.

FIG. 34 shows an illustration of the seventh step in a potential surgical installation method with the non-limiting implant shown. This step is to use the previously described first metacarpal drill guide. The guide 3400 is to be aligned with features on the side face of the artificial trapezium 3110, placing the drill guide in line with the transverse stem hole. A drill bit can then be used to create a percutaneous hole through the thumb and first metacarpal bone.

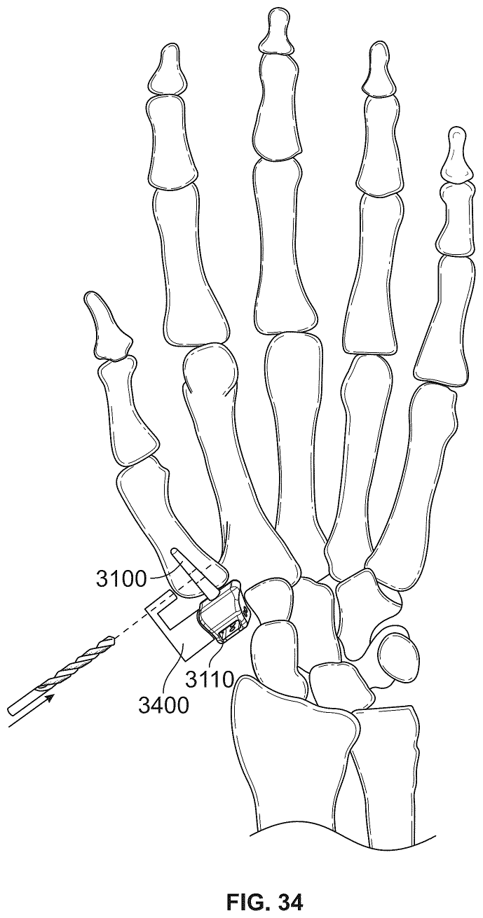

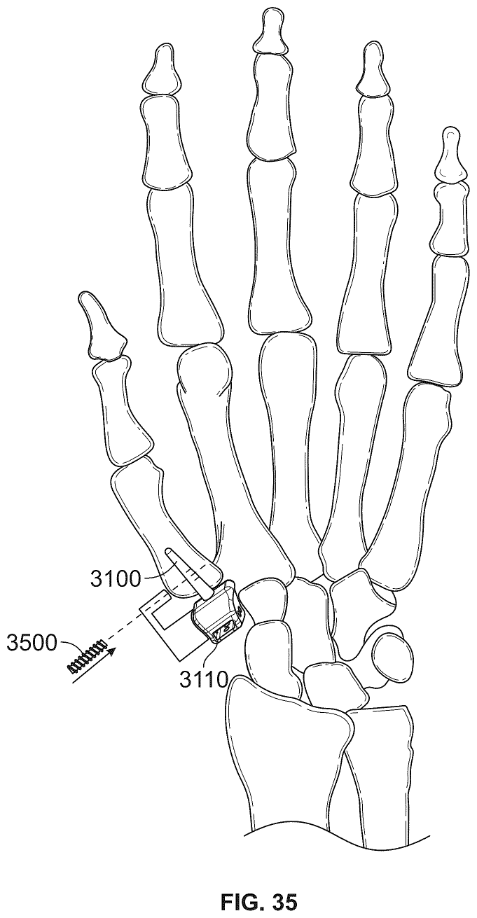

FIG. 35 shows an illustration of the eighth step in a potential surgical installation method with the non-limiting implant shown. In this step, a pin 3500, such as the illustrated screw, is inserted through the concentric holes to fix the stem 3100 in the first metacarpal.

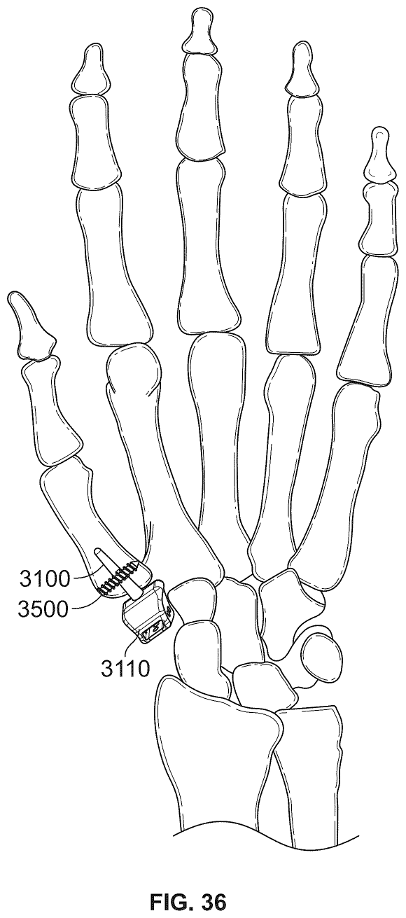

FIG. 36 shows an illustration of the ninth step in a potential surgical installation method with the non-limiting implant shown. In this step, the drill guide is removed and the pin 3500 is fully inserted, locking the stem from sliding or rotating in the first metacarpal.

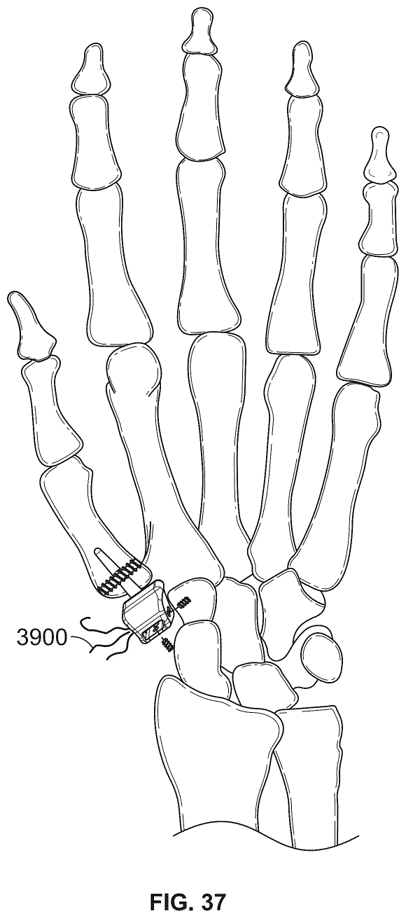

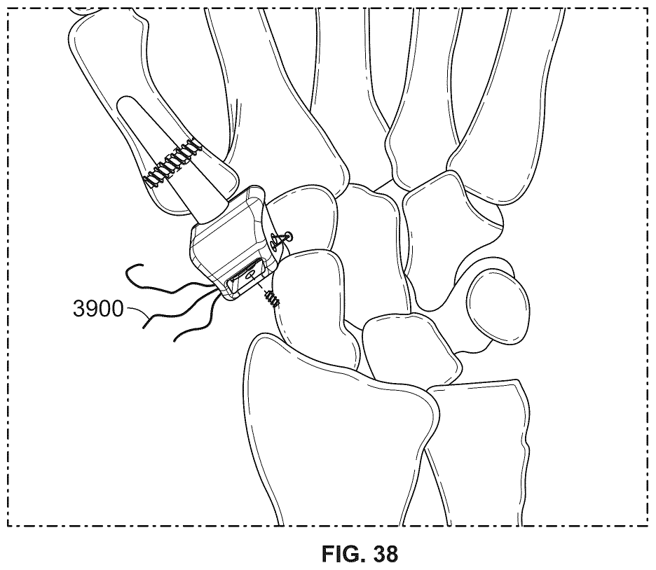

FIGS. 37 and 38 show an illustration of the tenth step in a potential surgical installation method with the non-limiting implant shown. In this final step, suture anchors 3900 are added to the scaphoid and trapezoid bones. This is just one of several potential fixation methods. Sutures are then run through the suture holes in the implant and, with the assistance of the window through the front and back faces, the sutures are tied to fix the implant in place.

### Alternate Design Iterations

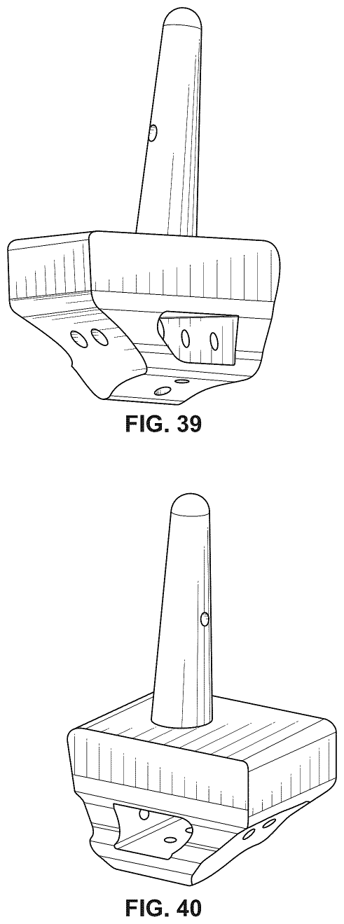

FIG. 39 shows a previous non-limiting design iteration of the implant. With similar features to other designs illustrated, this implant was better optimized for manufacturing via basic machining processes.

FIG. 40 shows an alternate view of a previous non-limiting design iteration of the implant. In this view, the side face that mates with the trapezoid bone can be seen. Suture holes for fixation are also present.

FIG. 41 shows an alternate non-limiting design iteration of the implant in which the stem and artificial trapezium are separate components. An extruded cylinder 4100 with a transverse pinhole 4110 is used to lock the artificial trapezium to the stem.

FIG. 42 shows an alternate non-limiting design iteration of the implant stem component. In this design, the stem is a modular design allowing for customization of stem length. In this figure, a base piece 4200 attaches to one design of the artificial trapezium using a hole 4210 with a locking pin that lines up with extrusion on the surface of the artificial trapezium.

FIG. 43 shows the second component 4300 of an alternate non-limiting modular stem design. This component is a male-to-female threaded spacer. The spacer is designed to thread onto a base component of the stem. Additional spacers can then be stacked in series to lengthen the stem to any desired length.

FIG. 44 shows the final component 4400 of an alternate non-limiting modular stem design. This final component is a cap to the stem, rounded at the top to adequately fill the entire space left by drilling a hole through the base of the stem.

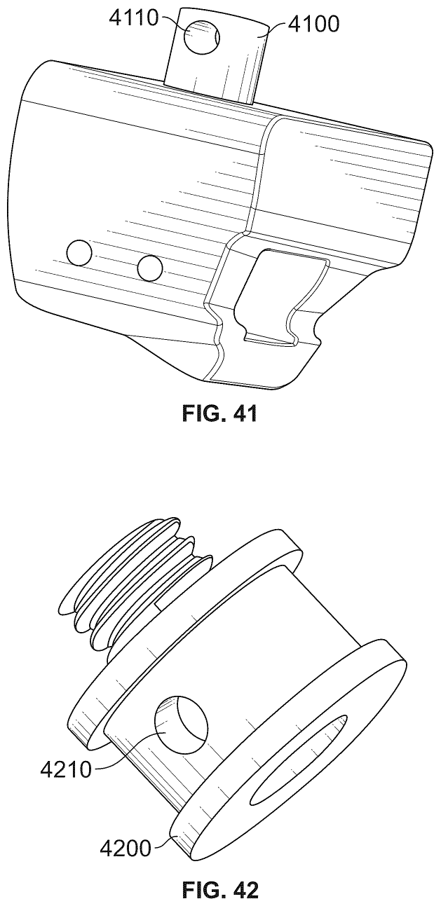

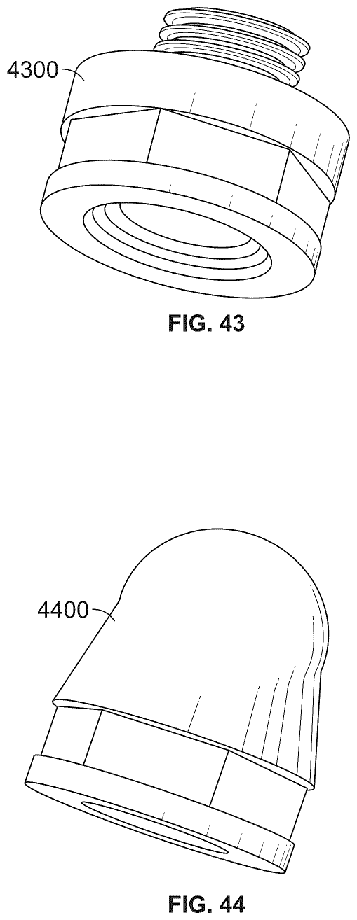

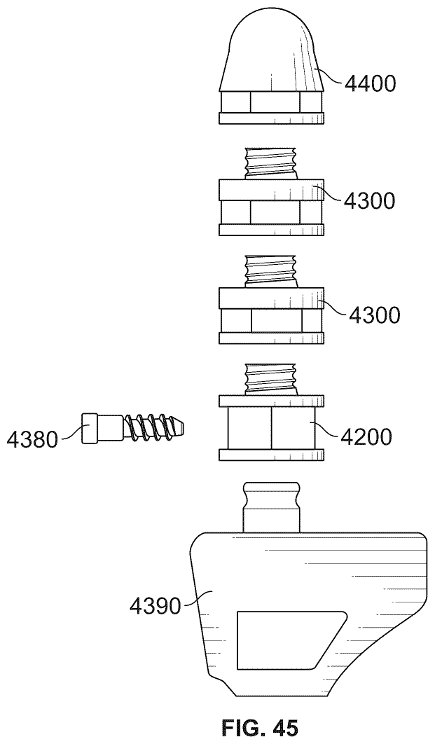

FIG. 45 shows an exploded assembly of an alternate non-limiting implant design that uses a previous implant design and a modular stem design. The modular stem components are assembled on the artificial trapezium in this illustration. A transverse screw 4380 is used to lock the stem assembly to the artificial trapezium 4390. Two spacer components are used in this illustration to achieve optimal stem length.

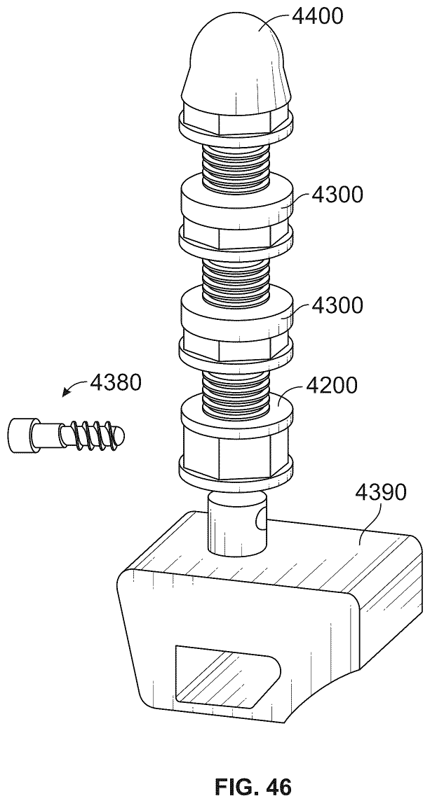

FIG. 46 shows a diametric exploded view of an alternate non-limiting implant design using a modular stem assembly.

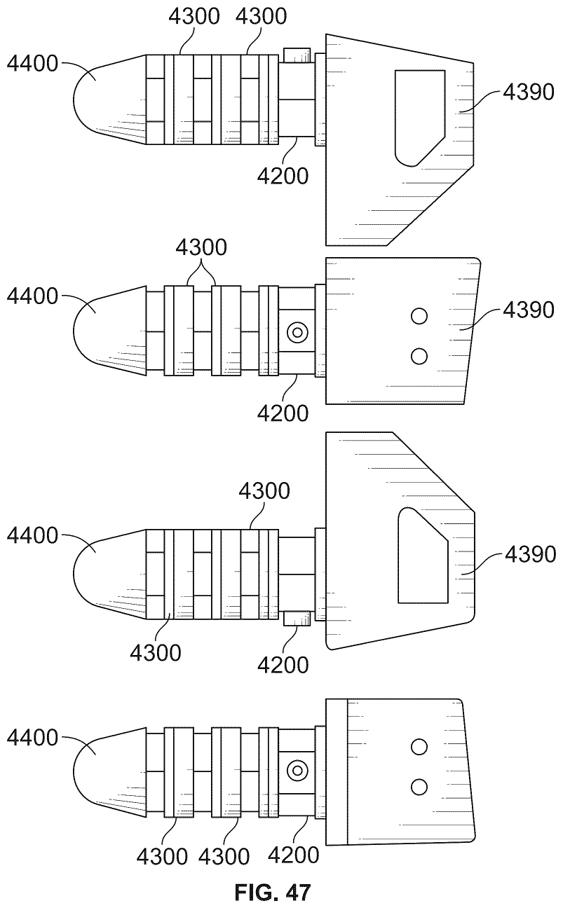

FIG. 47 shows front, back, and side views of an alternate non-limiting implant design. In this illustration, the modular stem is completely assembled. The stem extends perpendicularly from the artificial trapezium in these designs.

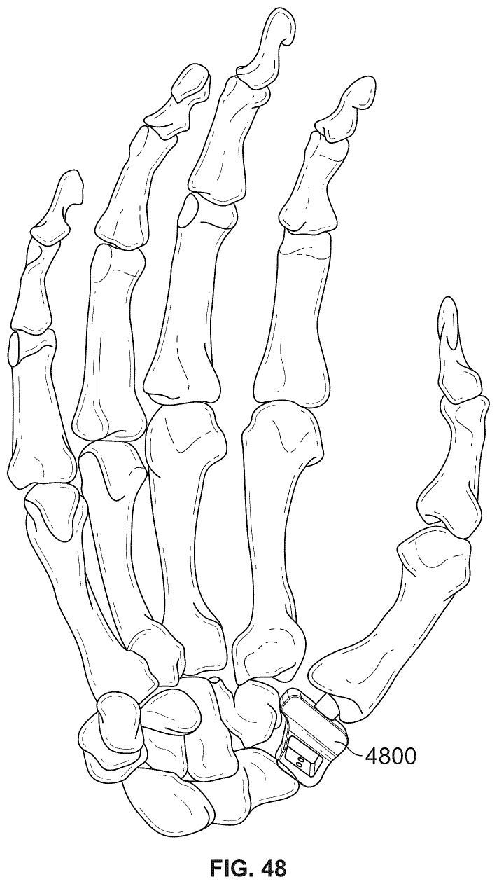

FIG. 48 shows the palmar surface of a hand skeleton with installed non-limiting implant design 4800.

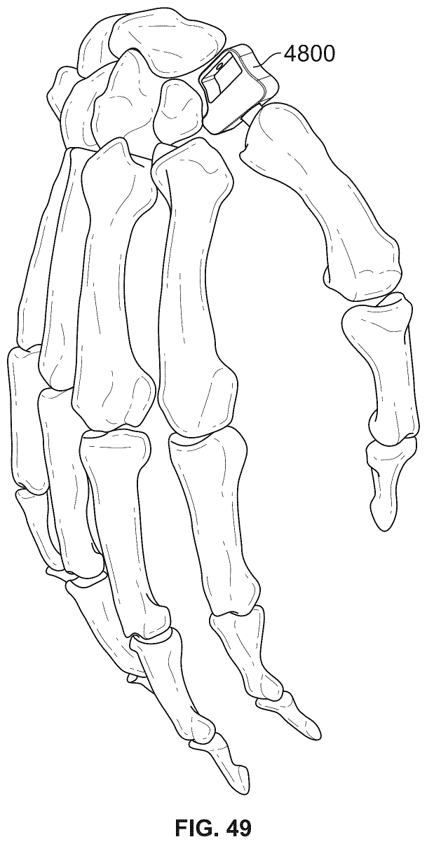

FIG. 49 shows the dorsal surface of a hand skeleton with installed non-limiting implant design 4800.

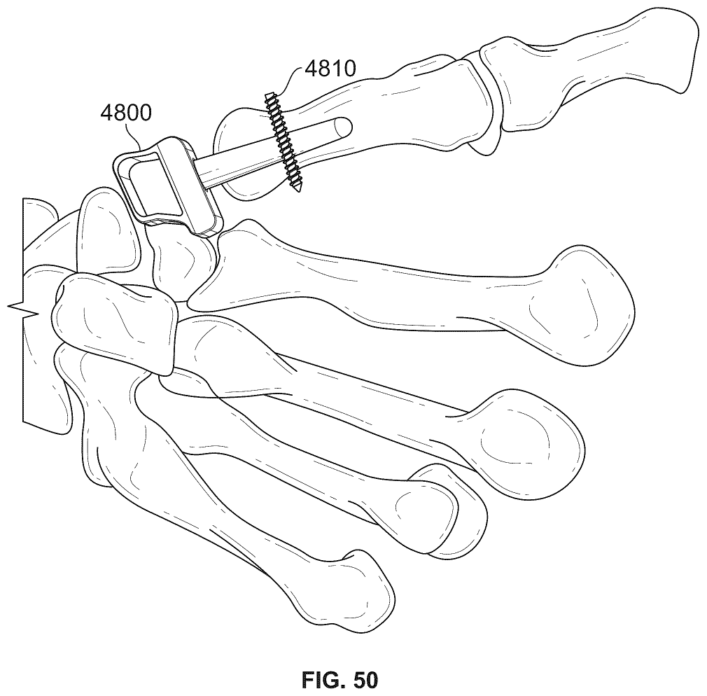

FIG. 50 shows the dorsal surface of an x-ray of a hand with installed non-limiting implant design 4800 and transverse screw/pin 4810.

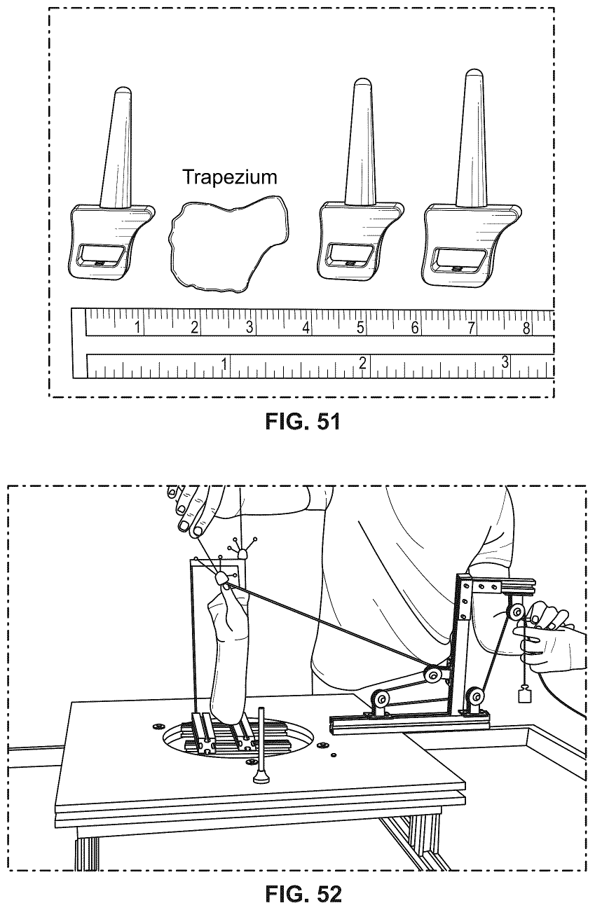

FIG. 51 shows a non-limiting size comparison of the implant used in experimental testing. Additionally, as previously stated, it is important to have access to a collection of prostheses in various sizes to accommodate diverse anatomies should a prosthesis be necessary. As a result, based on anatomical observation, one particular form of the present invention provides prostheses in at least three sizes, from the largest to the smallest.

FIG. 52 shows the experimental testing setup of the thumb range of motion. Positional displacement. Twelve fresh-frozen, male (6, mean age 43.19±8.64, mean weight 167.19±56.87) and female (6, mean age 45.64±10.35, mean weight 163.05±56.12) mid-humerus to fingertip were removed of second through fifth phalanges. All procedures were performed by a single, fellowship-trained, hand and wrist surgeon (NM). Left and right specimens were randomized for testing. One 3/32″ Steinmann pin was placed through the distal and proximal phalange, halfway through the first metacarpal. One 7/64″ Steinmann pin was placed ¾ through the second metacarpal. Specimens were placed in a custom testing apparatus that allowed for the evaluation of circumduction of the thumb. Each specimen was tested in six conditions: intact, fusion, fusion with partial resurrection, fusion with ligament transection, full trapeziectomy, and novel implant. Mechanical loading of 100 g was applied at the distal end of the first metacarpal to generate passive thumb circumduction at twelve angles incrementing 30° for a full revolution. Positional and rotational displacement data were collected using eight Optitrack motion capture cameras and Motive Body motion capture software. Two rigid body marker triads were used: axial placement along the first and second metacarpals. Each test was completed consecutively three times, at each angle. Data organization and calculations of angles for comparison were completed with MatLab software. Means, standard deviations, and 95% confidence intervals (CI) are reported. The remaining testing of specimens is underway to determine statistical significance.

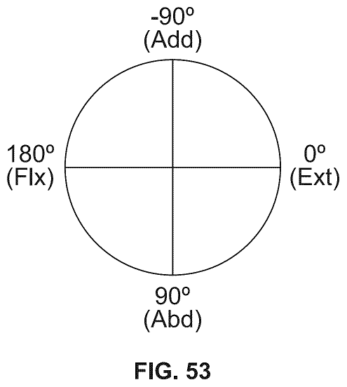

FIG. 53 shows an anatomical coordinate system for a right hand. Extension (Ext), Adduction (Add), Flexion (Flx), Abduction (Abd).

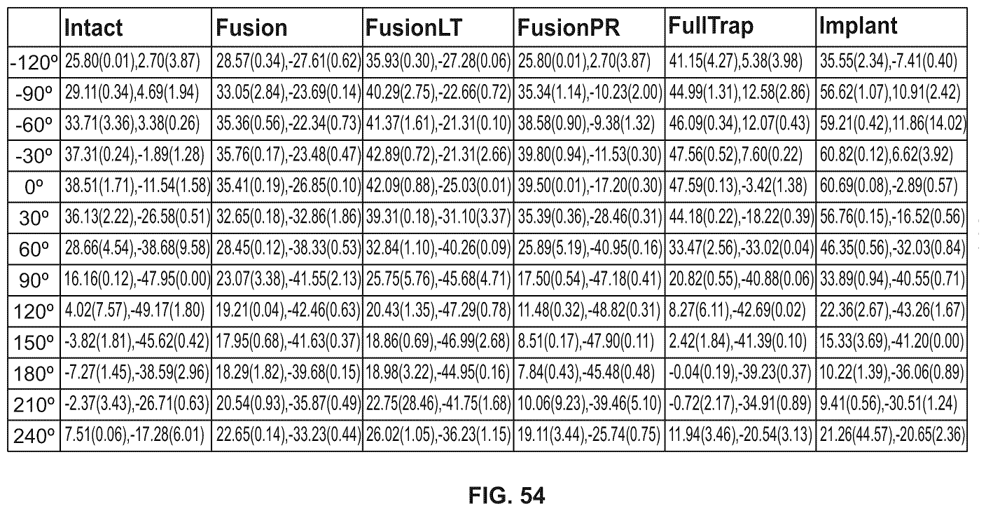

FIG. 54 shows the experimental data table. Mean, standard deviation, and 95% confidence intervals were quantified for intact, fusion, fusion with partial resurrection, fusion with ligament transection, full trapeziectomy, and novel implant at all angles of circumduction

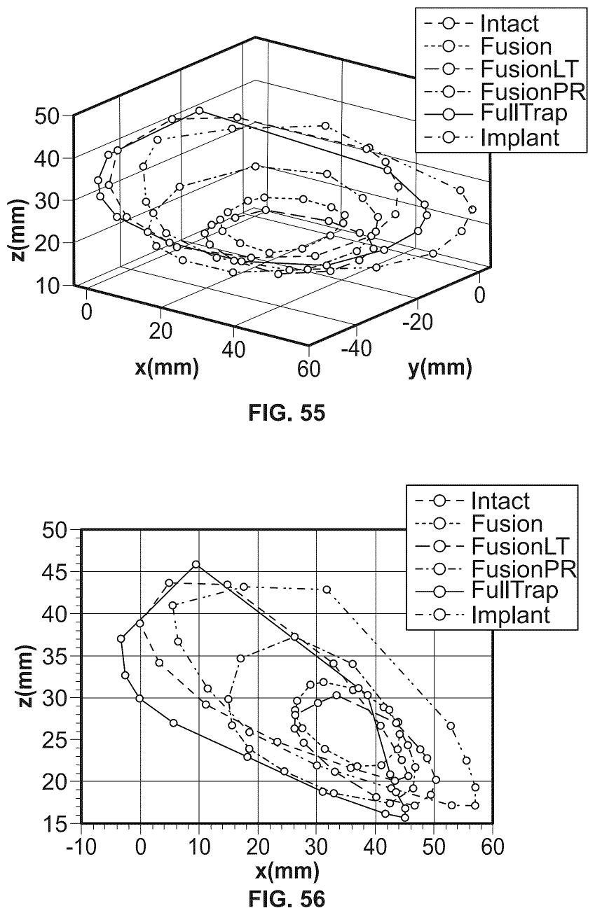

FIG. 55 shows a 3D view of the thumb's range of motion. Specimens were placed in a custom testing apparatus that allowed for the evaluation of circumduction of the thumb. Each specimen was tested in six conditions: intact, fusion, fusion with partial resurrection, fusion with ligament transection, full trapeziectomy, and novel implant. Mechanical loading of 100 g was applied at the distal end of the first metacarpal to generate passive thumb circumduction at twelve angles incrementing 30° for a full revolution.

FIG. 56 shows a xz view of the thumb's range of motion. Specimens were placed in a custom testing apparatus that allowed for the evaluation of circumduction of the thumb. Each specimen was tested in six conditions: intact, fusion, fusion with partial resurrection, fusion with ligament transection, full trapeziectomy, and novel implant. Mechanical loading of 100 g was applied at the distal end of the first metacarpal to generate passive thumb circumduction at twelve angles incrementing 30° for a full revolution.

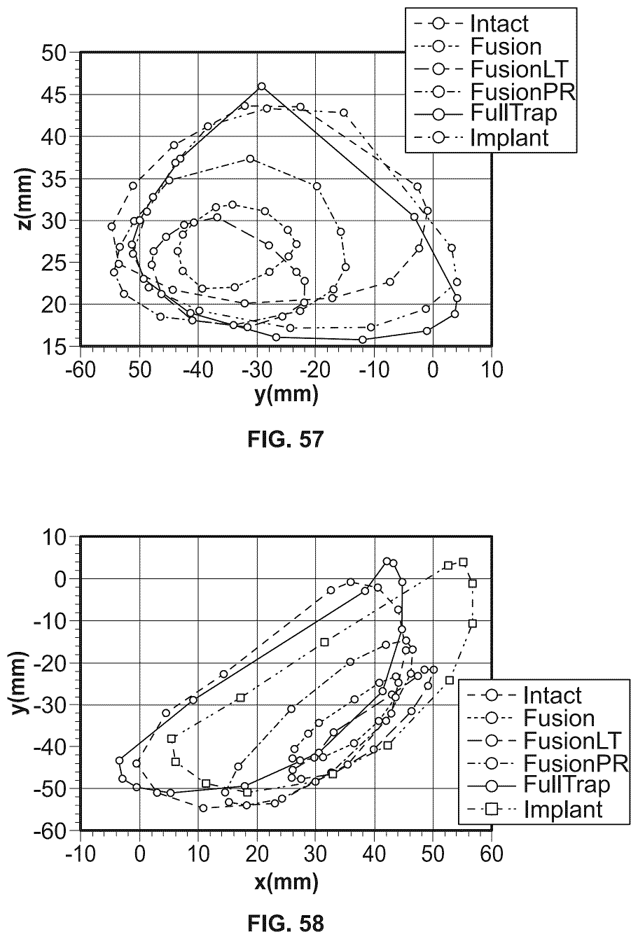

FIG. 57 shows a yz view of the thumb range of motion. Specimens were placed in a custom testing apparatus that allowed for the evaluation of circumduction of the thumb. Each specimen was tested in six conditions: intact, fusion, fusion with partial resurrection, fusion with ligament transection, full trapeziectomy, and novel implant. Mechanical loading of 100 g was applied at the distal end of the first metacarpal to generate passive thumb circumduction at twelve angles incrementing 30° for a full revolution.

FIG. 58 shows a xy view of the thumb's range of motion. Specimens were placed in a custom testing apparatus that allowed for the evaluation of circumduction of the thumb. Each specimen was tested in six conditions: intact, fusion, fusion with partial resurrection, fusion with ligament transection, full trapeziectomy, and novel implant. Mechanical loading of 100 g was applied at the distal end of the first metacarpal to generate passive thumb circumduction at twelve angles incrementing 30° for a full revolution.

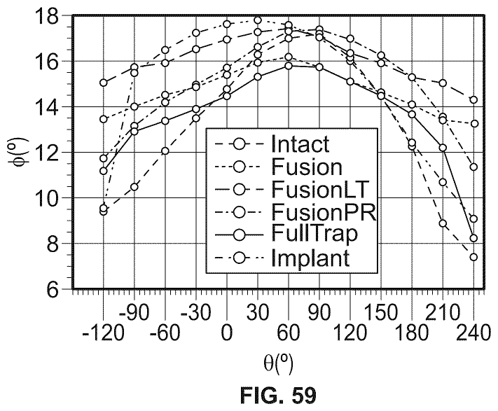

FIG. 59 shows the angular displacement of the thumb at each of the 12 angles. Specimens were placed in a custom testing apparatus that allowed for the evaluation of circumduction of the thumb. Each specimen was tested in six conditions: intact, fusion, fusion with partial resurrection, fusion with ligament transection, full trapeziectomy, and novel implant. Mechanical loading of 100 g was applied at the distal end of the first metacarpal to generate passive thumb circumduction at twelve angles incrementing 30° for a full revolution.

FIGS. 60A-60H shows how the stem locations on the top surface of the base may vary. Using the defined implant coordinate system representation of FIG. 61, the stem can move in the positive/negative x or y direction or a combination of both.

FIG. 61 shows varying angles of the stem to the base. α, θ, and φ are angles that determine the stem angle. The plane cuts the implant coordinate system representation into a positive half space where all angles exist. 0≤α≤180°, 0≤θ≤360°, 0≤φ¢≤180. The stem will always be inserted to be in line with the central axis of the intramedullary canal of the first metacarpal, but angling the stem relative to the base repositions the thumb, changing its position relative to the hand. The ability to change thumb position with the design angle of the stem angles allows for better patient outcomes.

FIG. 62 is an illustration of the fourth step in a potential surgical installation method with the non-limiting implant shown. A non-limiting temporary implant with a handle attached will be used to size the volume of the removed trapezium. The final size of the non-limiting implant will be chosen from the varying implant sizes in each surgical kit.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above-described embodiments, methods, and examples, but by all embodiments and methods within the scope and spirit of the disclosure.

1. An implant for replacing a trapezium bone in a human hand, the implant comprising:
a rigid body base sized and shaped to resemble the trapezium bone; and
a rigid body stem sized and shaped to be inserted inside a reamed first metacarpal along a central axis of an intramedullary canal of the reamed first metacarpal;
wherein the rigid body base includes:
a first curvature that is configured to fit an opposing surface of an adjacent trapezoid,
a second curvature that is configured to fit an opposing surface of a distal pole of a scaphoid, and
a third curvature that is configured to fit a saddle shape at a base of the reamed first metacarpal; and
wherein the rigid body stem is an attachable extrusion extending from the rigid body base for insertion into the reamed first metacarpal.

2. (canceled)

3. The implant of claim 1, wherein the rigid body stem is frusto-conical in shape.

4. The implant of claim 1, where the rigid body stem has at least one of a rough texture, a titanium plasma spray, or threads to improve fixation between the reamed first metacarpal and the rigid body stem.

5. The implant of claim 1, wherein the rigid body stem has a hole for a transverse screw or pin, and
wherein the rigid body stem is designed for press-fit within the reamed first metacarpal.

6. The implant of claim 1, wherein the rigid body stem has a round distal end and a proximal end that includes a locking mechanism.

7. The implant of claim 6, wherein the proximal end is angled so that the rigid body stem extends from the rigid body base at an angle to align with the central axis of the intramedullary canal of the reamed first metacarpal.

8. The implant of claim 3, wherein a distal end of the rigid body stem has a cross-sectional area that is smaller than a cross-sectional area of a proximal end of the rigid body stem.

9. The implant of claim 1, wherein the rigid body base includes a planar section that includes at least one of:
threads for attachment to the rigid body stem,
a cutout for receiving a portion of the rigid body stem, or
a post adapted to retain the rigid body stem.

10. The implant of claim 6, wherein the proximal end includes at least one of:
threads for attachment to the rigid body base, or
a locking mechanism to retain the rigid body stem to the rigid body base.

11. The implant of claim 6, further including a spacer located between the rigid body base and the rigid body stem.

12. The implant of claim 1, wherein the rigid body stem is comprised of multiple components including a base, a spacer, and a cap.

13. The implant of claim 1, wherein the rigid body base has a central opening for at least one of sutures, anchors, or guides.

14. The implant of claim 1, wherein the first curved surface includes first suture holes, and
wherein the second curved surface includes second suture holes.

15. The implant of claim 1, wherein the implant is a single mono-block.

16. (canceled)

17. The implant of claim 1, wherein the rigid body stem is positioned on an offset location on the rigid body base.

18. The implant of claim 1, wherein the rigid body stem is angularly positioned on the rigid body base.

19. (canceled)

20. (canceled)

21. An implant kit for replacing at least one trapezium, the implant kit comprising:
a plurality of bases including a first base and a second base,
wherein each base, of the plurality of bases, includes:
first curvature that is configured to fit an opposing surface of an adjacent trapezoid;
a second curvature that is configured to fit an opposing surface of a distal pole of a scaphoid; and
a third curvature that is configured to fit a saddle shape at a base of a first metacarpal; and
a plurality of stems including a first stem and a second stem,
wherein the first stem is a first attachable extrusion extending from a first location on the first base, and
wherein the second stem is a second attachable extrusion extending from a second location on the second base,
wherein the second location is different than the first location.

22. An implant kit for replacing a trapezium, the implant kit comprising:
a base and a plurality of stems;
wherein the base includes:
a first curvature that is configured to fit an opposing surface of an adjacent trapezoid;
a second curvature that is configured to fit an opposing surface of a distal pole of a scaphoid, and
a third curvature that is configured to fit a saddle shape at a base of a first metacarpal; and
wherein each stem of the plurality of stems is configured to attach to the base at a different angle.

23. The implant of claim 1, wherein a biocompatible material of the rigid body base and the rigid body stem includes at least one of titanium, stainless steel (SST), aluminum, polyetheretherketone (PEEK), cobalt chrome, polyethylene, polycarbonate, polyurethane, nylon, carbon fiber, other biocompatible metals and alloys, or other biocompatible polymers.

24. The implant kit of claim 21, wherein the first base and the first stem are designed specifically for right-hand anatomy, and
wherein the second base and the second stem are designed specifically for left-hand anatomy.
